SEQUENCE LISTING (14 sequences)

SEQ ID NO: 1 - protein, 5 aa, synthetic construct
TGGLS

SEQ ID NO: 2 - DNA, 21 bp, synthetic construct
  source 1..21: mol_type = other DNA
cactccctctctgcgcgctcg

SEQ ID NO: 3 - DNA, 22 bp, synthetic construct
  source 1..22: mol_type = other DNA
ggtggagcatgaattctacgtc

SEQ ID NO: 4 - DNA, 19 bp, synthetic construct
  source 1..19: mol_type = other DNA
gctctggttgttggtggcc

SEQ ID NO: 5 - DNA, 68 bp, synthetic construct
  source 1..68: mol_type = other DNA
  misc_difference 26: note = a, c, t, g, unknown, or other
  misc_difference 27: note = a, c, t, g, unknown, or other
  misc_difference 29: note = a, c, t, g, unknown, or other
  misc_difference 30: note = a, c, t, g, unknown, or other
  misc_difference 32: note = a, c, t, g, unknown, or other
  misc_difference 33: note = a, c, t, g, unknown, or other
  misc_difference 35: note = a, c, t, g, unknown, or other
  misc_difference 36: note = a, c, t, g, unknown, or other
  misc_difference 38: note = a, c, t, g, unknown, or other
  misc_difference 39: note = a, c, t, g, unknown, or other
  misc_difference 41: note = a, c, t, g, unknown, or other
  misc_difference 42: note = a, c, t, g, unknown, or other
  misc_difference 44: note = a, c, t, g, unknown, or other
  misc_difference 45: note = a, c, t, g, unknown, or other
ggccaccaacaaccagagcaccggtnnknnknnknnknnknnknnkggcttaagttccaccactgccc

SEQ ID NO: 6 - DNA, 22 bp, synthetic construct
  source 1..22: mol_type = other DNA
gcggaagcttcgatcaactacg

SEQ ID NO: 7 - DNA, 23 bp, synthetic construct
  source 1..23: mol_type = other DNA
cgcagagaccaaagttcaactga

SEQ ID NO: 8 - DNA, 27 bp, synthetic construct
  source 1..27: mol_type = other DNA
ttccacgtctttatatggtgcccagtc

SEQ ID NO: 9 - DNA, 10 bp, synthetic construct
  source 1..10: mol_type = other DNA
aatcagatac

SEQ ID NO: 10 - protein, 7 aa, synthetic construct
LSSTTAA

SEQ ID NO: 11 - protein, 7 aa, synthetic construct
DSRLSGT

SEQ ID NO: 12 - protein, 7 aa, synthetic construct
LEPDAAL

SEQ ID NO: 13 - protein, 7 aa, synthetic construct
IRWRATG

SEQ ID NO: 14 - protein, 7 aa, synthetic construct
RWPRRVL

(19) United States
(12) Patent Application Publication
(10) Pub. No.: US 2024/0404640 A1
(43) Pub. Date: Dec. 5, 2024

(54) MACHINE LEARNING GUIDED DESIGN OF VIRAL VECTOR LIBRARIES

(71) Applicants: CZ Biohub SF, LLC, San Francisco, CA (US); The Regents of the University of California, Oakland, CA (US)

(72) Inventors: David V. Schaffer, Danville, CA (US); Jennifer Listgarten, Berekeley, CA (US); DanQing Zhu, Oakland, CA (US); David Henry Brookes, Easton, MD (US)

(21) Appl. No.: 18/701,809

(22) PCT Filed: Nov. 2, 2022
(86) PCT No.: PCT/US2022/048736

§ 371 Date: Apr. 16, 2024

Related U.S. Application Data
(60) Provisional application No. 63/263,434, filed on Nov. 2, 2021.

(51) Int. Cl. G16B 40/20; G16B 30/00

(52) U.S. Cl. CPC G16B 40/20; G16B 30/00

(57) ABSTRACT

Various methods and systems are provided for designing viral vector libraries using machine learning models. In some embodiments, by training a machine learning model to predict a packaging fitness of a viral vector sequence, a viral vector library may be designed, wherein, for a desired library diversity, an increased packaging fitness may be achieved. In one example, a machine learning model may be trained to predict packaging fitness of a viral vector sequence by encoding the viral vector sequence as a feature set, mapping the feature set to a predicted packaging fitness of the viral vector sequence using a machine learning model, determining a loss based on a difference between a ground truth packaging fitness and the predicted packaging fitness of the viral vector sequence, and updating parameters of the machine learning model based on the loss.

### CROSS-REFERENCES TO RELATED APPLICATIONS

This application is a national phase application of PCT Application No. PCT/US2022/048736, filed Nov. 2, 2022, which claims priority to U.S. Provisional Application No. 63/263,434, entitled “Systems and Methods for Machine Learning Guided Design of Viral Vector Libraries” filed Nov. 2, 2021, the entire contents of which is herein incorporated by reference in its entirety for all purposes.

### REFERENCE TO AN ELECTRONIC SEQUENCE LISTING

The contents of the electronic sequence listing (110221-1435198-008010US SL.xml; size: 19,000 bytes; and Date of Creation: Jan. 4, 2023) is herein incorporated by reference in its entirety.

### BACKGROUND

Viral vectors, such as adenovirus, adeno-associated virus (AAV), retrovirus, and herpes simplex virus, hold tremendous promise as a gene delivery vector for gene therapy. Directed evolution has been applied as a powerful strategy to engineer biomolecules by generating large numbers of randomized variants and then selecting those with improved properties. A goal may be to select viral vector variants with improved properties, e.g., the ability to evade the immune system, or specificity for a particular tissue type. However, such testing and selecting of viral vector variants is very time consuming with many variants failing to improve properties. Therefore, it is desirable to improve the selection process.

### BRIEF SUMMARY

Embodiments of the subject matter disclosed herein relate to the design of viral vector libraries for providing gene therapies, e.g., to one or more specific types of cell. The inventors herein have at least partially addressed the above issues by developing systems and methods for predicting packaging fitness of viral vector sequences using machine learning models, and leveraging the predicted packaging fitness to design viral vector libraries with enhanced packaging viability at a given diversity.

A machine learning model (e.g., a supervised learning model) can be trained to predict fitness values for a property (e.g., packaging, cell sensitivity, cell specificity, and the like) of a particular viral vector using the sequence of the viral vector. In this manner, viral vectors with a beneficial fitness value (e.g., a high fitness value) for the property can be selected (at least on average), and viral vectors with a bad fitness may be excluded from a library used for downstream analysis. A training set can be generated experimentally, e.g., determine a ground truth fitness value for a set of viral vector sequences, which can include a sequence (e.g., N nucleotides long) inserted to promote packaging or other property. The machine learning model can be trained to reduce an error between a predicted fitness and the ground truth fitness.

The trained model can then predict the fitness for a new sequence, e.g., a combination of N nucleotides of an inserted sequence. An improved library can be obtained using sequences that have high fitness values. Such libraries can be obtained by sampling probability distributions of residues at variable locations in a viral vector sequence (constrained libraries) or by directly selecting viral vector sequences. In addition, a library can be designed such that it contains a diverse set of sequences. More diverse libraries will generally have lower fitness, so there is an inherent trade-off when targeting these two properties. Various libraries can be generated with different tradeoffs between average fitness values and diversity. Such libraries can be optimized to provide a highest fitness for a specific diversity, or vice versa.

In one example, a machine learning model may be trained to predict fitness values (e.g., packaging fitness values) of viral vector sequences by: selecting a training data pair comprising a viral vector sequence and a ground truth packaging fitness of the viral vector sequence, encoding the viral vector sequence as a feature set, mapping the feature set to a predicted packaging fitness of the viral vector sequence using a machine learning model, determining a loss based on a difference between the ground truth packaging fitness and the predicted packaging fitness of the viral vector sequence, and updating parameters of the machine learning model based on the loss.

The trained model may be employed to design viral vector libraries with an increased, or maximum, library fitness (e.g., average fitness for sequences in the library) at a desired degree of diversity. In one example, a viral vector library may be designed by: receiving a viral vector library encoding a plurality of viral vector sequences, determining an expected library fitness value of the viral vector library using a trained machine learning model, determining a diversity of the viral vector library, combining the expected library fitness of the viral vector library and the diversity to produce an objective score, and updating the viral vector library to increase the objective score. In this way, a bespoke viral vector library may be designed that trades a pre-determined amount of fitness to for a desired degree of diversity.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 shows a computing system, which may be used to execute one or more of the methods disclosed herein.

FIG. 2 shows an experimental workflow for generating pre- and post-packaged AAV5-7mer library data for ML-based library design.

FIG. 3 shows a table of example primer sequences for PCR reactions. FIG. 3 discloses SEQ ID NOS 3-5, 4, 6-8, and 7, respectively, in order of appearance.

FIG. 4A shows a comparison of models for predicting AAV5-7mer packaging log enrichment scores. FIG. 4B shows a similar plot to FIG. 4A except the comparison is of the use of weighted versus unweighted sequences during training, for the final selected model “NN, 100” and a baseline “Linear, Pairwise”.

FIGS. 5A-5B show schematic illustrations of the linear regression and neural networks models using the independent site (IS) representation for the input. FIGS. 5A-5B disclose “AATCAGATAC” as SEQ ID NO: 9.

FIG. 6 shows experimental titers (vg/μL) versus predicted log enrichment scores for five variants selected to span a broad range of predicted log enrichment scores. FIG. 6 discloses SEQ ID NOS 10-14, respectively, in order of appearance.

FIG. 7 is a flowchart illustrating a method for training a machine learning model to predict packaging fitness of viral vector sequences.

FIGS. 8A-8D show results for designed AAV5-based 7-mer insertion libraries, including a Pareto frontier (FIG. 8A) and probabilities for different distribution sets (FIGS. 8B-8D).

FIGS. 9A-9C shows a comparison of ML-designed libraries D2 and D3 to the NNK library.

FIG. 10 shows the entropy and mean predicted log enrichment for unconstrained libraries and constrained libraries corresponding to different settings of λ.

FIG. 11 shows a flowchart of an exemplary method of viral vector library design using a machine learning model.

FIG. 12A shows a general workflow of the primary adult brain infection study. FIG. 12B shows an effective number of variants (calculated from entropy) in NNK-post-brain infection vs. D2-post-brain infection. FIG. 12C shows entropy (diversity) comparison between synthesized NNK and ML-designed D2 libraries after packaging and infection of primary adult brain tissue.

FIG. 13A shows empirical probabilities distribution of each amino acid at each position for D2 post-packaging and post-brain infection. FIG. 13B shows NNK marginal probabilities of amino acids at each position after packaging and primary brain selection.

FIG. 14 shows scatterplots illustrating the behavior of individual variants over packaging and primary brain selection highlighting the top 20% of total reads in each library.

FIG. 15 shows scatterplots of individual variants' (log) prevalence after packaging and primary brain selections, displaying and highlighting variants in the top 50% and 80% of total reads in each library.

FIG. 16 illustrates a measurement system 1600 according to an embodiment of the present disclosure.

FIG. 17 shows a block diagram of an example computer system usable with systems and methods according to embodiments of the present disclosure.

### TERMS

A library can be a specific set of unique sequences (referred to as a sequence set) or a probability distribution, where a probability distribution at a given position includes respective probabilities for any possible biological sequence (of nucleotides, amino acids) in its domain. Such a distribution may be factored into a set of distributions (also referred to as a distribution set), one per sequence position. Alternatively, the distribution may be captured by any parametric or non-parametric distribution, such as a Hidden Markov Model, a Variational Auto Encoder, a Diffusion generative model, or a neural network. Each sequence in the sequence set can be derived from the distribution set by randomly sampling (e.g., using Monte Carlo techniques) each residue of the sequence according to the probability distribution for that sequence. Thus, a sequence set can be randomly sampled from a distribution. A sequence set can include a specified number of sequences (e.g., ten million) that is less than all possible combinations of residues in a sequence, e.g., as not all sequences can be tried since 4.4 trillion sequences are possible for a 21 nt sequence.

A distribution over sequences can be defined by the product of the probability of each residue at each site in a sequence, e.g., for each nt at each site of a 21 nt sequence or for each amino acid at each site of a peptide with length 7. In the nucleotide example, a distribution would have 84 probability parameters (4×21). If a library was defined using amino acids, the distribution could include 140 probabilities (20×7), where 20 is the number of possible amino acids.

A fitness value may refer an experimentally measured property (e.g., packaging) of the viral vector sequence that relates to an ability to deliver a gene therapy to a cell. A fitness value is a numerical amount as opposed to a binary variable. And a numerical difference can be determined between two fitness values (e.g., a ground truth fitness value and a predicted fitness value). A library has an overall fitness, e.g., an average fitness or other statistical value. A library fitness value can indicate a collective value (e.g., an average, median, mode, etc.) for the library, which may be measured for the entire library (e.g., a total number of particles before and after an experiment, such as a packaging experiment) or determined from individual values for each unique sequence. An expected value of the fitness of a distribution can be determined by sampling from the distribution and calculating the average fitness among the samples (also referred to as a Monte Carlo approximation). A degree of enrichment between a pre- and post-packaging library can be used as a packaging fitness.

A “machine learning model” (ML model) can refer to a software module configured to be run on one or more processors to provide a classification or numerical value of a property of one or more samples. Machine learning models may be defined by “parameter sets,” including “parameters,” which may refer to numerical or other measurable factors that define a system (e.g., the machine learning model) or the condition of its operation. Example machine learning models may include different approaches and algorithms including analytical learning, artificial neural network, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm. Further examples include linear regression, logistic regression, convolutional neural network (CNN), deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein.

A “supervised learning model” is an ML model that is trained using a training set having known values/labels (e.g., fitness values, such as packaging fitness values). In “supervised learning,” during a training phase, a machine learning model can learn correlations between features contained in feature vectors and associated labels. After training, the machine learning model can receive unlabeled feature vectors and generate the corresponding labels. For example, during training, a machine learning model can evaluate labeled viral sequences, then after training, the machine learning model can evaluate unlabeled viral sequences, in order to determine the fitness (e.g., packaging fitness) of a viral sequence. In some cases, training a machine learning model may include identifying the parameter set that results in the best as measured using a “loss function,” which may refer to a function that relates a model parameter set to a “loss value” or “error value,” a metric that relates the performance of a machine learning model to its expected or desired performance. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

A “sequence read” or “read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example PCR techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000 or 50,000 or 100,000 or 500,000 or 1,000,000 or 5,000,000 sequence reads, or more, can be analyzed. Such sequences can be used in a training set.

### DETAILED DESCRIPTION

The genetic code of viruses can be modified for delivering gene therapy. Viruses, such as adeno-associated viruses (AAVs), hold tremendous promise as delivery vectors for clinical gene therapy, but they need improvement. AAVs with enhanced properties, such as more efficient and/or cell-type specific infection, can be engineered by creating a large, diverse starting library and screening for desired phenotypes, in some cases iteratively. A library of different modified viruses can be analyzed experimentally to identify a viral sequence that perform the best.

But many individual viral variants experimental fail, resulting in extra time and money. For example, although this approach has succeeded in numerous specific cases, such as infecting cell types from the brain to the lung, the starting libraries often contain a high proportion of variants unable to package their genomes (e.g., assembling protein into a capsid shell and loading the viral genome into the shell). Thus, such experimental time-consuming failures are often due to the virus not packaging into functional capsids or package their genomes after random mutagenesis. It is also challenging to design libraries comprising novel and diverse viral vector sequences, while retaining the functional ability of the library to package DNA payloads. Therefore, it is generally desired to design viral vector libraries that are diverse, yet have a high probability of providing a desired property, such as assembling into functional capsids and successfully packaging their genomes. In this manner, an improved library can have a higher proportion of candidate viral sequences that will be functional (e.g., successful packaging) for an increased chance of success in downstream implementation.

Embodiments can improve one or more overall properties of a library (e.g., number of variants that package) by inserting sequences into the viral vector sequence. A set of sequences can be selected to provide a library with optimized properties. Other example properties/characteristics that can be improved by inserting a sequence of a specified number of residues include vector tropism (targeting of specific cell populations), transduction efficiency (ability to enter cells), immune-evasion (ability to circumvent neutralizing antibodies), transgene expression (ability to enter the nucleus and uncoat transgene cassette to express), and tissue penetration (ability to bio-distribute widely, including passing through blood-brain-barrier). The ability of a sequence to provide a property can be represented as a fitness value, which can be experimentally determined and ultimately predicted.

The present disclosure describes a machine learning (ML)-based method for systematically designing more effective starting libraries, e.g., ones that have broadly good packaging capabilities. Diversity of a starting library can also be a factor. Some embodiments can optimize for a particular property (e.g., packaging) and diversity, e.g., with a particular weighting scheme over how much one is favored over the other; this may be done by selecting a percentage weighting for the property (e.g., packaging) and diversity terms for any loss (cost) function used in determining an optimal starting library.

Such carefully designed but general libraries stand to significantly increase the chance of success in engineering any property of interest. As an example, we use this approach to design a clinically-relevant AAV peptide insertion library that achieves 5-fold higher packaging fitness than the state-of-the-art library, with negligible reduction in diversity. We demonstrate the general utility of this designed library on a downstream task to which our approach was agnostic: infection of primary human brain tissue. The ML-designed library had approximately 10-fold more successful variants than the current state-of-the-art library. Not only are such new libraries useful for any number of other engineering goals, but our library design approach itself can also be applied to other types of libraries for AAV and beyond.

As part of designing an improved library, a machine learning model can be trained to predict the desired property, e.g., packaging. Such a model can predict the experimental property of any viral sequence (e.g., when the model is trained for a desired length of insertion sequence), and an optimal set of viral sequences can be selected for the library. Accordingly, systems and methods can predict packaging fitness of viral vector sequences, and designing viral vector libraries, using machine learning models.

To prepare the training set for training such a prediction model, the experimental property (to be used as the label in the training set) can be measured experimentally, e.g., by sequencing or probe-based (e.g., PCR) techniques. For example, an initial set of viral vector sequences can be synthesized and then experimentally analyzed to the experimental property. For example, a number of reads for each unique sequence can be determined by sampling the initial set and by sampling a set resulting from a physical process (e.g., packaged into functional virions, as may be done using the packaging cell line HEK293T) corresponding to the experimental property. When packaging is the property, successfully packaged viral particles can be harvested from the packaging cell lines. Successfully packaged particles can be recovered, and the genomes can be extracted for analysis. A ratio of the read count from the initial set and the post-packaging set can provide a fitness value (enrichment score) that can be used as the label (ground truth) for the training set.

Deep sequencing technologies allow thousands to hundreds of millions of sequences to be assayed in parallel, enabling large-scale probing of fitness landscapes of viral vector sequences. Such data can be used to train supervised machine learning (ML) models that predict viral properties from an input of a sequence. Such a prediction model can guide the ML-guided viral vector library design, which can enable generation of capsid libraries that balance library diversity with overall packaging fitness of the library. Such informed and optimal library creation can set the stage for downstream use of these libraries for therapeutic end goals.

Machine learning (ML) augmented approaches described in this disclosure can inform the randomization of the proteins in the starting pool of directed evolution for the therapeutically relevant domain of adeno-associated virus (AAV)-based gene delivery system. By doing so, we are able to dramatically improve the overall “fitness” of the starting pool of variants, and consequently, also the ability to achieve desired AAV variants—in particular those that can infect human brain cells, a therapeutic target of interest.

### I. Generation of a Viral Vector Library

While naturally-occurring AAVs can be clinically administered safely and in some cases efficaciously, they have a number of shortcomings that limit their use in many human therapeutic applications. For example, naturally-occurring AAVs do not target delivery to specific organs or cells, their delivery efficiency is limited, and they are susceptible to pre-existing neutralizing antibodies [1-3]. Consequently, directed evolution of the AAV capsid protein has emerged as a powerful strategy for engineering therapeutically suitable or optimal AAV variants. In directed evolution, a diversified library of AAV capsid sequences is subjected to multiple rounds of selection for a specific property of interest, with the aim of identifying and enriching the most effective variants [1, 4]. Primary techniques for constructing AAV starting libraries include error-prone PCR [1, 5], DNA shuffling [6, 7], structurally-guided recombination [8], peptide insertions [9], and phylogenetic reconstruction [10]. Recent studies have also explored computational strategies for setting the parameters that control the construction of these libraries. For example, genomic junctions that minimize AAV structure disruptions, suitable for recombination libraries, were computationally identified [9]. For mutagenesis libraries, genomic locations and their mutation probabilities were identified using single-substitution variant data, or by way of ancestral imputation from phylogenetic analysis [10-11].

Although successes have been achieved with directed evolution [4-6,8-9,13], several challenges are slowing progress [12]. For instance, a substantial fraction of the variants in the starting libraries for these selections are unable to assemble properly or package their payload efficiently [11,14-15]. Consequently, much of the library is wasted, thereby decreasing the chance of successfully achieving any desired engineering goal in the downstream selections. Next Generation Sequencing (NGS) technologies enable analysis of properties for individual variants within a library, such as packaging fitness and infectivity, and the large quantity of data resulting from such assays and machine learning (ML) can be a useful tool to help design more effective starting libraries for directed evolution. Herein, we propose methods to design such a ML-guided library, e.g., that balances the requirements of packaging and diversity, to improve the probability of success in any general AAV directed evolution goal.

Embodiments can systematically navigate an optimal trade-off between diversity and packaging. Various approaches herein can (i) allow for the use of any predictive model of fitness, (ii) explicitly address and control the diversity within the designed library, and (iii) be broadly applicable to different kinds of library construction.

### A. Insertion of Sequence

As an example, we instantiated and evaluated our library design approach by designing a 7-mer peptide insertion library for AAV serotype 5 (AAV5) to improve packaging, and optionally to optimally balance diversity and overall packaging fitness. Among the natural AAV serotypes, AAV5 has been suggested as a promising candidate for clinical gene delivery because of the low prevalence of pre-existing neutralizing antibodies and successful clinical development for hemophilia B [21-24]. We focus, specifically, on peptide insertion libraries because they are both simple and highly practical, having already been translated to the clinic (e.g., NCT03748784, NCT04645212, NCT04483440, NCT04517149, NCT04519749, NCT03326336, NCT05197270) [25]. Other kinds of libraries can also be used, e.g., where the diversity is spread across the entire cap gene—such as error prone PCR, recombination, and ancestral libraries. Such libraries may require long read sequencing, e.g., single molecule sequencing as may be provided by nanopore sequencing or single-molecule real-time (SMRT) sequencing.

To achieve the 7-mer peptide insertion, a nucleotide sequence corresponding to the 7-mer peptide sequence can be inserted into the viral capsid sequence to improve packaging. In one example, 21 nucleotides are inserted into the viral genome, corresponding to 7 codons (7 amino acids)) in the capsid protein sequence. Sequences of other lengths (e.g., 5, 6, 7, 8, 9, etc.) can be used, and amino acid linkers of various lengths (e.g., 4, 5, 6, etc.) can be used. A set of training sequences are generated; such training sequences would correspond to different combinations of the 21-nt sequence. Such a set would typically not include all possibilities of the 21-nt sequence since that would be too many to practically generated. The training set may be ordered from a vendor, e.g., an NNK library (described below). The training sequences may effectively be randomly selected (e.g., as part of a stochastic biochemical process) based on probability distributions (e.g., NNK distribution) for the proportions for different nucleotides at each position in the 21-nt sequence, e.g., by using the desired proportion of bases for each new position. These training sequences are synthesized and their ground truth packaging fitness can be determined experimentally, e.g., as the relative increase in sequence counts after transfection.

B. Library with Uniform Probability Distribution

One example of a viral vector library is constructed from 7 concatenated copies of the NNK degenerate codon (NNK)₇. The “NNK” moniker refers to a broadly used strategy [28-30] involving a uniform distribution over all four nucleotides (N) in the first two positions of a codon, and equal probability on nucleotides G and T (K) in the third position; where the K in the third position was chosen to reduce the chance of stop codons which typically render the protein non-functional. More specifically, the NNK codon specifies the marginal probability distribution where every nucleotide/residue is equally likely in the first two positions of the codon (Pᵣ(A)=Pᵣ(T)=Pᵣ(C)=Pᵣ(G)=0.25), and in the final position Adenine and Cytosine have zero probability, while Thymine and Guanine are each equally probable (Pᵣ(T)=Pᵣ(G)=0.5). Each of the 7 amino acids in the insertion is sampled at random from this distribution during library construction.

Using the NNK degenerate codon in this way is one approach for generating viral vector libraries, as it induces a distribution of amino acids where each amino acid has a non-zero probability but with minimal probability of stop codons. While the NNK library exhibits high diversity, the majority of sampling sequences will likely fail to package into viable capsids due to potential structural destabilization of the capsid, which yields a low packaging fitness for the resulting library, reducing the efficiency for downstream screening processes. Some embodiments disclosed herein improve upon such an approach, by providing a mechanism for designing viral vector libraries with an enhanced packaging fitness at a desired level of diversity.

In some example techniques described in more detail below, we used libraries with a variable 7-amino acid (7-mer) NNK sequence inserted at position 575-577 in the viral protein monomer, within a loop at the 3-fold symmetry axis associated with receptor binding and cell-specific entry [26, 27].

Although NNK libraries are among the most promising AAV libraries [2], a substantial fraction (>50%) of the variants in these libraries fail to package (i.e., do not package into viable capsids, and many more have lower packaging fitness than the parental virus [14, 15]. For example, placing a large hydrophobic residue in the 7-mer (solvent-exposed) region is likely destabilizing. Much of the experimental library is thus effectively wasted on poor fitness variants.

C. Library with Optimal Probability Distribution

A goal was to improve upon the NNK library and implicitly uncover a broad set of rules, as yet unknown, for insertion sequences that confer higher packaging fitness and then encode them in a library design so as to avoid such problems. In some embodiments, our design approach can specify probabilities for each nucleotide in each position of the codon, at each position in the 7-mer, in a manner that achieves better overall packaging than NNK, while maintaining high diversity. For example, an implementation might specify for the first codon that the first nucleotide in the codon should be chosen with 20% chance as an A, 40% chance as a C, 35% chance as a T and 5% G; then specify four other such probabilities for the other two positions in the codon, for a total of 12 specified values. A designed library can specify these 84 (=7×12) probabilities, which in turn will dictate the mean packaging fitness—through a complicated relationship that will be approximated with our machine learning predictive model—and library sequence diversity. We refer to designed libraries specified in this way as position-wise nucleotide specified.

To generate the training data set for training the prediction model to predict packaging fitness for any given viral sequence, we assess the packaging fitness for variants in a library, e.g., a NNK library. We then use these estimated packaging efficiencies as labels to build the prediction model from peptide insertion sequence to packaging fitness. We also develop a design approach that can systematically trade off library diversity with packaging fitness, enabling us to choose an optimal trade-off.

Such an approach to ML-guided library design biases library construction towards variants that package well, thereby reducing the amount of wasted sequences and space in screening tasks. We show that our design approach yields a library with 5-fold higher packaging fitness than the NNK library, with negligible sacrifice to diversity, suggesting that our library will be more generally useful. As further evidence, when we subjected the NNK library to one round of packaging selection—after extracting sequences that successfully packaged, re-package and measure the titer—the resulting pool of variants still had a lower packaging fitness than that of our initially designed library, while also being substantially less diverse.

To demonstrate the general downstream utility of our designed library on an engineering task for which it was not designed (primary human brain tissue selection), the ML-guided library yielded a 10-fold higher number of infectious variants compared to the NNK library, and these variants can be further selected for efficient and cell-specific infectivity. While we focus on a therapeutically relevant capsid 7-mer peptide insertion library, our methods are general and can be applied to other AAV library types, and to proteins beyond AAV.

### II. Machine Learning System for Library Design

The machine learning models described herein can be executed on a computer system. The computer system can include the modules for the machine learning prediction model and the library design and can store the training data.

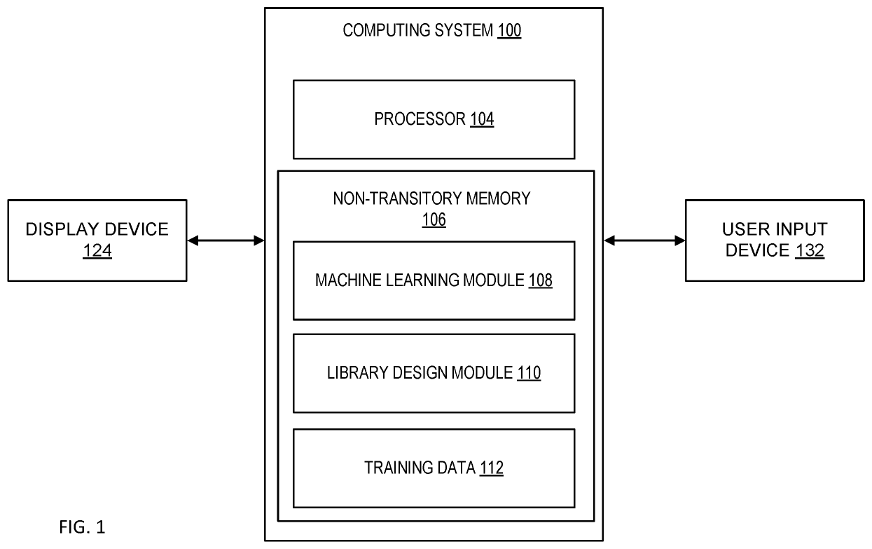

FIG. 1 shows a computing system, which may be used to execute one or more of the methods disclosed herein. Computing system 100 includes a user input device 132, and display device 134. In some embodiments, computing system 100 may receive sequencing data and/or fitness data (e.g., packaging fitness data or other property to be optimized) from a remote device, wherein the remote device is communicably coupled to computing system 100 via wired or wireless connection.

Computing system 100 includes a processor 104 configured to execute machine readable instructions stored in non-transitory memory 106. Processor 104 may be single core or multi-core, and the programs executed thereon may be configured for parallel or distributed processing. In some embodiments, the processor 104 may optionally include individual components that are distributed throughout two or more devices, which may be remotely located and/or configured for coordinated processing. In some embodiments, one or more aspects of the processor 104 may be virtualized and executed by remotely-accessible networked computing devices configured in a cloud computing configuration.

Non-transitory memory 106 may store machine learning module 108, library design module 110, and training data 112. Machine learning module 108 may include one or more machine learning models, comprising a plurality of parameters. In some embodiments, the machine learning models stored in machine learning module 108 may include linear models, and/or neural networks. In one example, machine learning module 108 stores a plurality of weights, biases, activation functions, pooling functions, and instructions for implementing a neural network to map a feature set extracted from a viral vector sequence to a packaging fitness. The packaging fitness can indicate a degree of enrichment between a pre and post packaging library.

In some embodiments, the machine learning module 108 includes instructions that, when executed by the processor 104, extract features from a viral vector sequence to produce a feature set. In some embodiments, machine learning module 108 may comprise one or more trained or untrained machine learning models. In some embodiments, machine learning module 108 may include instructions for executing one or more gradient descent algorithms to train the model. Machine learning module 108 may further include one or more loss functions, whereby a loss for a machine learning model may be determined based on a predicted packaging fitness and a ground truth packaging fitness.

In some embodiments, the machine learning module 108 is not disposed at the computing system 100, but is disposed at a remote device communicably coupled with computing system 100 via wired or wireless connection. Machine learning module 108 may include various machine learning model metadata pertaining to the trained and/or un-trained machine learning models stored thereon. In some embodiments, the machine learning model metadata may include an indication of the training data used to train a machine learning model, a training method employed to train a machine learning model, and an accuracy/validation score of a trained machine learning model. In some embodiments, machine learning module 108 may include metadata for a trained machine learning model indicating a type of viral vector sequence for which the model is trained to predict packaging fitness.

In some embodiments, machine learning module 108 may include instructions for training a machine learning model by executing one or more of the operations of methods described herein. In one embodiment, the machine learning module 108 includes one or more gradient descent algorithms, loss functions, and machine executable instructions for generating and/or selecting training data for use in training a machine learning model.

Non-transitory memory 106 further includes library design module 110, which comprises machine executable instructions for optimizing a viral vector library design based on a desired tradeoff between diversity and packaging fitness. Library design module may include instructions, that when executed by processor 104, perform one or more of the operations of methods described herein. In some embodiments, the library design module 110 is not disposed at the computing system 100, but is disposed remotely, and is communicably coupled with computing system 100.

Non-transitory memory 106 may further include training data 112, comprising a plurality of training data pairs. Each training data pair can include a viral vector sequence (from which features may be extracted) and a ground truth viral vector characteristic (e.g., a packaging fitness value measured experimentally). In one example, the plurality of training data pairs may be used in conjunction with a training method to train a machine learning model to predict one or more sequence characteristics, such as a packaging fitness, based on a viral vector sequence.

Computing system 100 may further include user input device 132. User input device 132 may comprise one or more of a touchscreen, a keyboard, a mouse, a trackpad, a motion sensing camera, or other device configured to enable a user to interact with and manipulate data within computing system 100. In some embodiments, user input device 132 enables a user to adjust a desired diversity of a viral vector library.

Computing system 100 includes display device 134. In some embodiments, display device 134 may comprise a computer monitor. Display device 134 may be configured to receive data from computing system 100, and to render the data as a graphical display. Display device 134 may be combined with processor 104, non-transitory memory 106, and/or user input device 132 in a shared enclosure, or may be a peripheral display device and may comprise a monitor, touchscreen, projector, or other display device known in the art, which may enable a user to view images, and/or interact with various data stored in non-transitory memory 106.

It should be understood that computing system 100 shown in FIG. 1 is for illustration, not for limitation. Another appropriate computing system may include more, fewer, or different components.

### III. Example Workflow for Packaging and Library Design

As an example, we developed a ML-based method for systematically designing diverse AAV libraries with good packaging capabilities, so that they can be used as starting libraries in directed evolution for engineering specific and enhanced AAV properties. An example workflow can (i) synthesize and sequence a baseline NNK library, the pre-packaged library; (ii) transfect the library into packaging cells (i.e., HEK 293T) to produce AAV viral vectors, harvest the successfully packaged capsids, extract viral genomes, and sequence to obtain the post-packaging library used to determine the fitness values corresponding to the viral vector sequences; (iii) build a supervised model (e.g., using regression) where the target variable reflects the packaging fitness of each insertion sequence in the pre-packaged library; (iv) systematically invert the predictive model to design libraries that trace out an optimal trade-off curve between diversity and fitness; and (v) select a library design with a suitable tradeoff. We then validated both the predictive model and the designed library by experimentally measuring library packaging success and sequence diversity. We also demonstrate that our ML-designed library is better able to infect primary human brain tissues as compared to the baseline NNK library.

Such techniques improve upon the NNK library and implicitly uncover a broad set of rules, as yet unknown, for insertion sequences that confer higher packaging fitness and then encode them in our library design so as to avoid such problems. In particular, our design approach can specify probabilities for each nucleotide in each position of the codon, at each position in the 7-mer, in a manner that achieves better overall packaging than NNK, while maintaining high diversity.

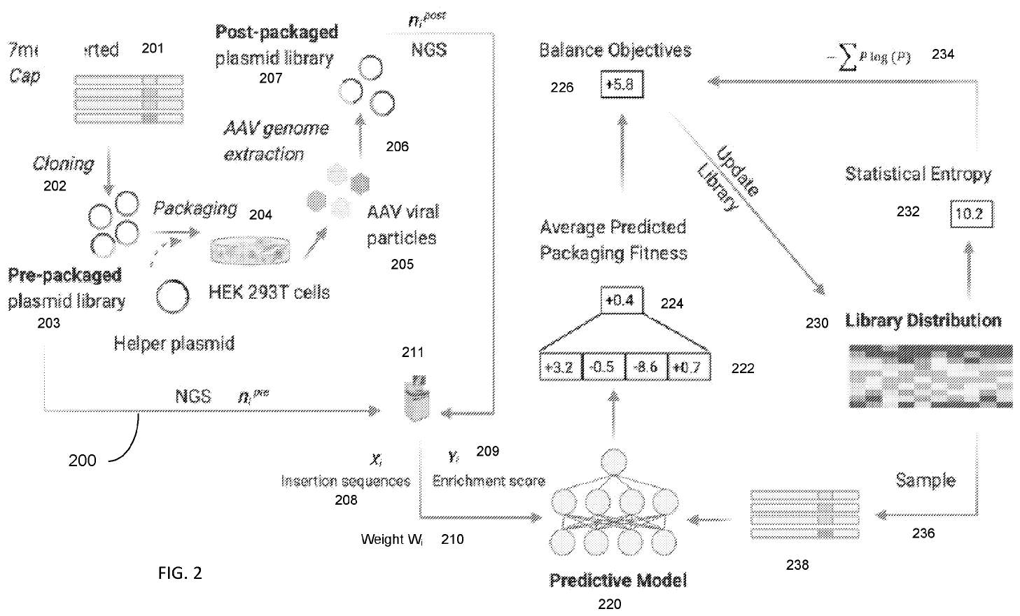

FIG. 2 shows an experimental workflow for generating pre- and post-packaged AAV5-7mer library data for ML-based library design. NGS is next-generation sequencing; n_{I} is the number of reads for each unique insertion sequence i, either before or after packaging. Other techniques besides sequencing can be used to quantify the sequences (before and after packaging), such as digital PCR (e.g., digital-droplet PCR) or real-time PCT. Experimental data was used to build a supervised regression model where the target variable reflects the packaging success of each insertion sequence. The predictive model was then systematically inverted to design libraries that trace out an optimal trade-off curve between diversity and packaging fitness.

### A. Generation of Training Set

In an example implementation for generating training data for the models, (i) a NNK library (e.g., a 7mer peptide library) can be used (ii) a set of sequences (e.g., 7mers) sampled from the NNK probability distribution can be synthesized, (iii) the library can be inserted into viral genomes, (iv) the resulting set of viral genomes can be sequenced to get “pre-selection” counts, (v) a packaging experiment can be implemented, and (vi) the sequences of the viruses that successfully packaged to get “post-selection” counts can be sequenced. The sequences that were observed in the pre-selection and post-selection pools can be used as training data.

Referring to FIG. 2, process 200 generates the training set of viral vector sequences and their measured enrichment scores (fitness values). Process 200 can provide insertion sequences Xᵢ and enrichment scores Yᵢ that are used to train a predictive model 220 (also referred to as a prediction model), which may be any suitable supervised model that can predict a numerical fitness value (as opposed to a binary classification) such as regression model.

At step 201, a 21-mer nucleotide sequence (corresponding to the 7-mer peptide) is inserted into the viral sequences to synthesize a plurality of distinct viral vector sequences. The selection of which sequences to insert can follow a probability distribution. In one example, roughly 10⁷ variants (unique sequences) from the NNK library were synthesized. In various implementations, synthesizing the plurality of viral vector sequences may include generating 10⁷-10⁹ capsid-modified variants with 21 random nucleotides (corresponding to seven random amino acids) inserted by overlap splicing polymerase chain reaction (PCR), followed by ligation reaction and electroporation transformation.

At step 202, a plurality of plasmids encoding capsid-modified variants are created. A sub-section of the native viral vector sequence includes the mutant/variant sequence that was inserted. The plasmids of the viral vector sequences can be cloned to increase the raw number initial viral vector sequences.

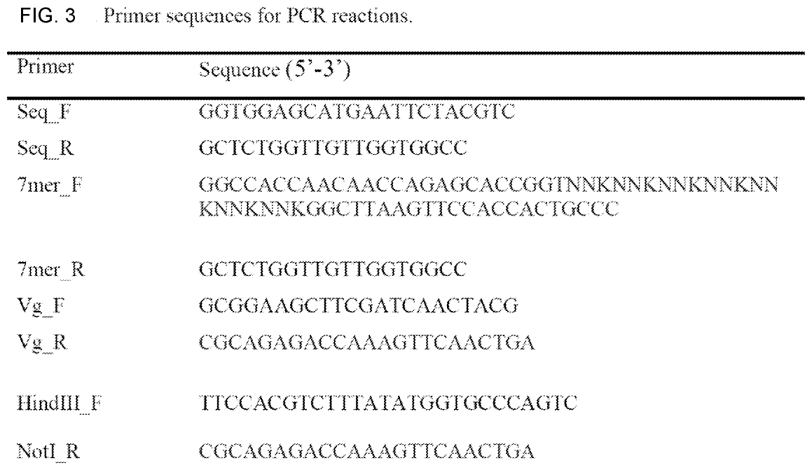

At step 203, the plasmid library was then packaged to yield the NNK pre-packaged library. As an example for constructing the pre-packaged library, libraries with a variable 7-amino acid (7-mer) insertion region flanked by amino acid linkers (TGGLS (SEQ ID NO: 1)) can be introduced at position 575-577 in the viral protein monomer. (NNK)₇ oligo can be synthesized (Elim) and introduced to the 5′ end of the right fragment by a primer overhang. Left and right fragments can be each PCR amplified by primers Seq_F/Seq_R and 7mer_F/7mer_R, respectively (FIG. 3). PCR products of the two fragments can then be purified individually and proceeded to the overlap extension PCR using Vent DNA polymerase (Thermofisher) with equimolar amounts of the left and right fragments for a total of 250 ng DNA templates. The resulted library can then be digested with HindIII and NotI and ligated into the replication competent AAV packaging plasmid pSub2. The resulting ligation reaction can be electroporated into Escherichia coli for plasmid production and purification. Replication competent AAV can then be packaged as been described previously [1,26].

At step 204, the resulting pre-packaged library may then transfected into an expression cell-line, such as by using polyethylenimine (PEI) to transfect the viral vector sequences into HEK293T cells to produce viral vector proteins. As an example, AAV library vectors can be produced by triple transient transfection of HEK293T cells with the addition of the pRepHelper, purified via iodixanol density centrifugation, and buffer exchanged into PBS by Amicon filtration.

At step 205, the resulting viral particles were harvested and purified. After a specified duration post-transfection (e.g., 72 hours), the expressing cells may be harvested, lysed, and the virus particles may be purified. In one example, the established iodixanol protocol may be used to lyse and purify the expressed viral vectors. Instead or if additional selection is desired (e.g., for other properties), there can be another step of ‘selection’, for example, infection to specific cell types, incubation with evading antibodies, etc. For example, if screening for enhanced infectivity of a given target cell, embodiments can compare the number of reads of a given variant that successfully made it to that cell divided by the number of times that read appears in the virus that was administered to the cells.

As example embodiments, packaged AAV vectors can be combined with equal volume of 10× DNase buffer (New England Biolabs, B0303S) and 0.5 μL 10 U/μL Dnase I (New England Biolabs, M0303L) incubate for 30 min at 37° C. Then equal volume of 2× Proteinase K Buffer can be added with sample to break open capsid. After heat inactivating for 20 min at 95° C., the sample can be further diluted at 1:1000 and 1:10,000 and use as templates for titer. Dnase-resistant viral genomic titers can be measured using digital-droplet PCR (BioRad) using with Hex-ITR probes (CACTCCCTCTCTGCGCGCTCG (SEQ ID NO: 2)) tagging the conserved regions of encapsidated viral genome of AAV.

At step 206, the AAV genomes were extracted, yielding the NNK post-packaged library 207. Successfully packaged capsid sequences may be recovered and measured, to determine a post packaging abundance of each viral vector sequence. In one example, Hirt viral genome extraction and PCR may be used to sequence/measure the abundance of successfully packaged viral vector sequences. The packaged viral vector sequences may be collectively referred to as the post-packaging library.

In another example, segments inserted into the viral vector sequences may be selectively PCR amplified and deep sequenced. A sequence platform (e.g., an Illumina NovaSeq 6000 platform) may be used to selectively amplify and sequence the pre- and post-packaging libraries to obtain a first pre-packaging abundance and a second post-packaging abundance of each of the plurality of viral vector sequences. Accordingly, the sequences from both pre- and post-packaged libraries can PCR amplified and deep sequenced to determine nᵢ^{pre} (number of pre-packaged copies of a unique sequence) and nᵢ^{post} (number of post-packaged copies of the unique sequence). In one implementation, these experiments yielded 49,619,716 pre-packaged and 55,135,155 post-packaged sequence reads, which collectively yielded read counts for 8,552,729 unique peptide sequences. Note that only 218,942 of the 8,552,729 unique sequences appear in both the pre- and post-selection libraries.

At 211, pairs of insertion sequences and enrichment scores are combined to provide training data pairs. For each unique insertion sequence Xᵢ 208, we used the pre- and post-read counts to calculate an enrichment score Yᵢ 209, which may be a log score. Enrichment score Yᵢ 209 can include a ratio of nᵢ^{post}/nᵢ^{pre}. The enrichment score Yᵢ 209 is a measure of its packaging fitness. Since different insertion sequences can generate a same amino acid sequence, such duplicative insertion sequences can be combined when determining the enrichment score. Thus any individual nt sequence of a family of nt sequences that generate the same amino acid sequence can have the same enrichment score. In this manner, predictive model 220 can receive a peptide insertion sequence as opposed to a nucleotide insertion sequence.

In some embodiments, each unique sequence can be treated the same when training the predictive model. That is the error for any sequence affects the loss function the same. However, a variant (unique sequence) that appeared in 10 pre- and 100 post-packaged sequencing reads would have the same enrichment score as one that appeared in 1 and 10 sequencing reads, even though the former has more data to support its value (i.e., is more stably statistically estimated).

Accordingly, some embodiments can treat different sequences differently. This may be done using a weight Wᵢ 210 for each unique sequence. We derived a procedure to take into account the different statistical stability when estimating model parameters (e.g., regression parameters). One implementation assigns a weight to each unique sequence that is higher when the statistical estimate is more stable; higher weighted sequences have more influence on predictive model 220. For example, a first variant with a read count ratio of 10:1 would get a smaller weight than a second variant with a ratio 100:10, as the former provides weaker evidence of enrichment. For example, the weight of the first variant can be 1 and the weight of the second variant can be 10. A variant having a particular read count (pre- or post-packaging) can be assigned any weight (e.g., at random) and the weights of other variants can be scaled according to their respective read counts. Example techniques to determine the weight are provided below.

### 1. Measuring Post-Packaging Sequences

In one implementation, after primary tissue infection, capsid sequences can be recovered by PCR from harvested cells using primers HindIII_F and NotI_R. A ˜75-85 base pair region containing the 7mer insertion was PCR amplified from harvested DNA. Primers included the Illumina adapter sequences containing unique barcodes to allow for multiplexing of amplicons from multiple libraries. PCR amplicons were purified and sequenced with a single read run-on Illumina NovaSeq 6000.

Each read contained (i) a 5 bp unique molecular identifier, (ii) a fixed 21 bp primer sequence, (iii) a 6 bp sequence representing the pre-insertion linker (two fixed amino acids that connect the insertion sequence to the capsid sequence at position 587), (iv) a variable 21 bp sequence containing the nucleotide insertion sequence, and (v) a 9 bp representing the post-insertion linker (three fixed amino acids that connect the insertion sequence to the capsid sequence at position 588). We filtered the reads, removing those that either contained more than 2 mismatches in the primer sequences or contained ambiguous nucleotides. After this filtering, the pre- and post-libraries contained 46,049,235 and 45,306,265 reads, respectively. The insertion sequences were then extracted from each read and translated to amino acid sequences.

### 2. Fitness Value and Weights (Log Enrichment Score and Variance)

A fitness value can be determined as an enrichment score. In some embodiments, a log enrichment scores (Equation 1) can be determined for each insertion sequence using the (filtered) sequencing data to quantify each sequence's effect on packaging. In some embodiments, an enrichment score may be used as a ground truth packaging fitness and may be estimated according to the below equation:

$${y}_{i} = log \frac{{n}_{i}^{post}}{{n}_{i}^{pre}} - log \frac{{N}^{post}}{{N}^{pre}} , \qquad ( Eqn . 1 )$$

where yᵢ is the ground truth packaging fitness of the viral vector sequence i, nᵢ^{pre} is the first abundance of the viral vector sequence measured before the packaging process, nᵢ^{post} is the second abundance of the viral vector sequence measured after the packaging process, N^{pre} is a first total abundance of viral vector sequences measured before the packaging process, and N^{post} is a second total abundance of viral vector sequences measured after the packaging process. The packaging fitness, yᵢ, may also be referred to herein as an enrichment score, or enrichment factor.

The second term above can provide a normalization across different samples, e.g., to account for different total abundances in the pool. The may be for a variety of reasons for different total abundances. For instance, some samples may have more cells, which can provide an overall higher gain, but the second term can subtract off the overall enrichment for all sequences, so the relative increase for individual sequences can be determined. A pseudo-count of 1 can be added to each count so that the log enrichment score could still be calculated when the sequence appeared in only one of the libraries. The natural log or other logs can be used.

A variance of a fitness value can be used to determine the weight of a given sequence for use in the loss function, as described herein, e.g., where the weight is the inverse variance. The variance associated with each log enrichment score can be determined using Equation 2, which follows by noting that each of the raw counts associated with an enrichment score is a random variable. Specifically, the count associated with a sequence can be modeled as a Binomial random variable [32]. The log enrichment score is then the log ratio of two Binomial random variables; it can be shown with the Delta Method [36] that, in the limit of infinite samples, the log ratio of two Binomial random variables converges in distribution to a Normal random variable with mean and variance approximated by Equations 1 and 2, respectively [32, 33].

$${σ}_{i}^{2} = \frac{1}{{n}_{i}^{p o s t}} ( 1 - \frac{{n}_{i}^{p o s t}}{{N}^{p o s t}} ) + \frac{1}{{n}_{i}^{p r e}} ( 1 - \frac{{n}_{i}^{p r e}}{{N}^{p r e}} ) , \qquad ( Eqn . 2 )$$

where nᵢ^{pre} is the first abundance of the viral vector sequence measured before the packaging process, nᵢ^{post} is the second abundance of the viral vector sequence measured after the packaging process, N^{pre} is a first total abundance of viral vector sequences measured before the packaging process, and N^{post} is a second total abundance of viral vector sequences measured after the packaging process. The total abundance of viral vectors can be used to normalize the variance across different samples as different amounts of overall enrichment can depend on specific experimental properties, such as the number of cells used in the transfection.

### B. Training and Evaluation of Predictive Models

Predictive model 220 can be trained using each unique insertion sequence Xᵢ 208 and the corresponding enrichment score Yᵢ 209 (or other fitness value if a different sequence property/characteristic) is determined, and potentially the corresponding weight Wᵢ 210. Predictive model 220 can be trained using a loss function that is a function of a difference (e.g., squared or absolute difference) between the predicted packaging fitness value and the ground truth packaging fitness value (e.g., enrichment score Yᵢ 209), which is determined experimentally. The numerical fitness values are numerical amounts from which the numerical difference is determined. The parameters of predictive model 220 can be determined using any suitable optimization technique to minimize the loss function. Example techniques are described herein, such as gradient descent, conjugate gradient, and Newton techniques, as well as any randomization techniques (e.g., Monte Carlo or simulated annealing) to find a global minimum.

As mentioned above, the difference (e.g., squared error) term for each viral sequence can be weighted based on the number of initial sequences or post-packaging sequences. In one example, the squared difference can be weighted by an estimate of the noise in the fitness measurement, which can include both the pre- and post-selection sequencing counts. See Eqn. 5 below. For the same enrichment, sequences with larger numbers of read counts can be weighted higher, as such sequences would likely have less variation and have fitness values that are more reliable.

### 1. Comparison of Predictive Models

To find a model type to use for our ML-guided library design, we compared seven classes of ML regression models: three linear models and four feed-forward neural networks (NNs). Each model was trained using the log enrichment scores as the target variable and using the sequence-specific weights described above for determining the loss value of the loss function as a weighted sum of the log enrichment scores.

The three linear models differed in the set of input features used. One used the “Independent Site” (IS) representation wherein individual amino acids in each k-mer (e.g., a 7-mer) insertion sequence were one-hot encoded (i.e., 0 or 1 for each position for the 20 amino acids for a human). Similarly, individual nucleotides in each 21-mer nt insertion can be one-hot encoded. Another used a “neighbors” representation comprised of IS features and additionally pairwise interactions between all positions that are directly adjacent in the amino acid sequence. Pairwise interactions can also be one hot encoded, where at each pair of positions, there are 20\hat{ }=400 possible pairs of amino acids. Each (L choose 2) pair of positions has a length 400 vector one-hot encoded vector associated with it that indicates the pair of amino acids at those positions. The pairwise interactions can also be defined for nucleotides. In either case, a given interaction pair can be defined using an interaction vector where only one element has ‘1’ corresponding to the actual two types of residues. Thus, each pair can be represented. For the “neighbors” representation, encoding the viral vector sequence as a feature set can comprises, encoding each pair of adjacent (neighboring) residues of the viral vector sequence as a vector to produce a plurality of interaction vectors.

The third used a “pairwise” representation comprised of the IS features and additionally all pairwise interactions among all positions in the sequence. The encoding for the “neighbors” and “pairwise” representation can comprise encoding each pair of adjacent residues (neighboring residues) of the viral vector sequence as a vector to produce a plurality of interaction feature vectors. The pairwise representation can encode each residue-residue interaction of the viral vector sequence to produce a plurality of interaction vectors. As examples, other encodings can be from another machine learning model or based on physico-chemical properties.

All neural network models used the IS features alone, as these models have the capacity to construct higher-order interaction features from the IS features. Each NN architecture comprised exactly two densely connected hidden layers with tanh activation functions, although more hidden layers and different activation functions (e.g., softmax, sigmoid, ReLU, etc.) can be used. The four NN models differed in the size of the hidden layers, with each using either 100, 200, 500, or 1000 nodes in both hidden layers.

We compared the performance of these seven models using the standard (unweighted) Pearson correlation between model predictions and true log enrichment scores on a held-out test set (training with weighted samples as described earlier). We randomly split the data into a training set containing 80% of the data points and a test set containing the remaining 20% of the points. Because a goal was to design a library of sequences that package well, we also studied how the models' predictive accuracy changed when restricted to sequences in the test set with observed high packaging log enrichment. Specifically, we computed the Pearson correlation on subsets of the test set restricted to the fraction K of sequences with the highest observed log enrichment. By varying K, we traced out a performance curve where for lower K, the evaluation is more focused on accurate prediction of higher log enrichment scores rather than lower ones.

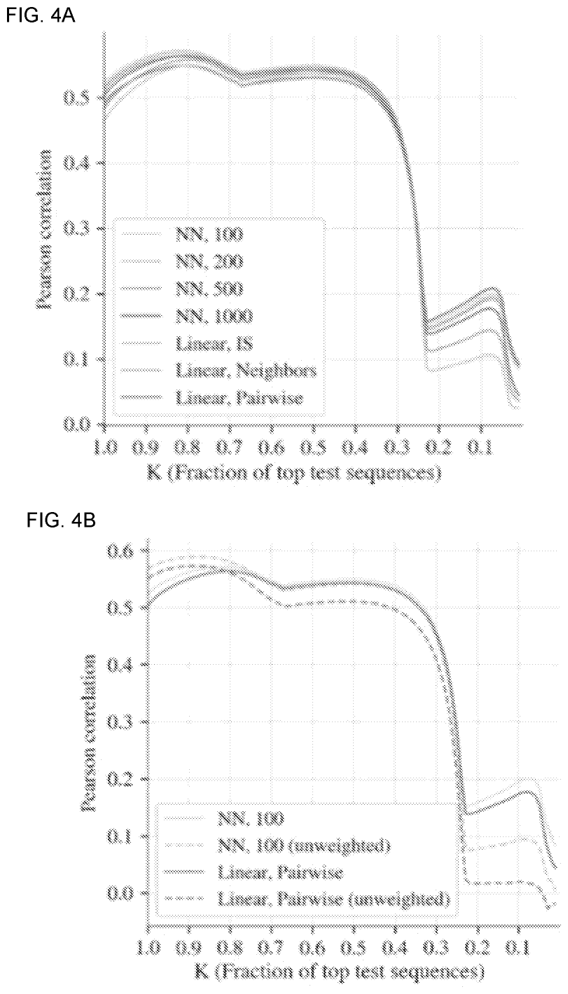

FIG. 4A shows a comparison of models for predicting AAV5-7mer packaging log enrichment scores. The correlation is between predicted and true log enrichment scores. K denotes what fraction of top-ranked observed log enrichment test sequences were used. Each point along each curve (computed at intervals of 0.01 on the horizontal axis) represents the Pearson correlation between the predicted and true enrichment scores of viral vector sequences in a culled subset of the test set. The weighted loss function is used.

Overall, we found that the NN models performed better than the linear models, presumably owing to their capacity to construct more complex functions—in particular, to capture higher-order epistatic interactions in the fitness function. We selected “NN, 100” as our final model, as it performed similarly to the overall best-performing model, “NN, 1000”, but with many fewer parameters.

Next, we assessed the effect of training with our sequence-specific weights by retraining two of the models—the final model, “NN, 100” model and the “linear, Pairwise” model—this time with all weights set to 1.0 (i.e., unweighted), again using Pearson correlation to evaluate.

FIG. 4B shows a similar plot to FIG. 4A except the comparison is of the use of weighted versus unweighted sequences during training, for the final selected model “NN, 100” and a baseline “Linear, Pairwise”. Training in this unweighted manner (i.e., using the weights for the loss function based on read counts), rather than weighted, resulted in a performance benefit for K near 1.0, but degraded the performance near K<0.25, a regime of particular interest since it focuses on variants with high log enrichment, and we ultimately aim to design a library that packages well (i.e., with high enrichment). We ranked the sequences from low to high, so the top % (<0.25) are the high enriched ones. These results further supported our choice of the weight trained “NN, 100” model with which to do library design.

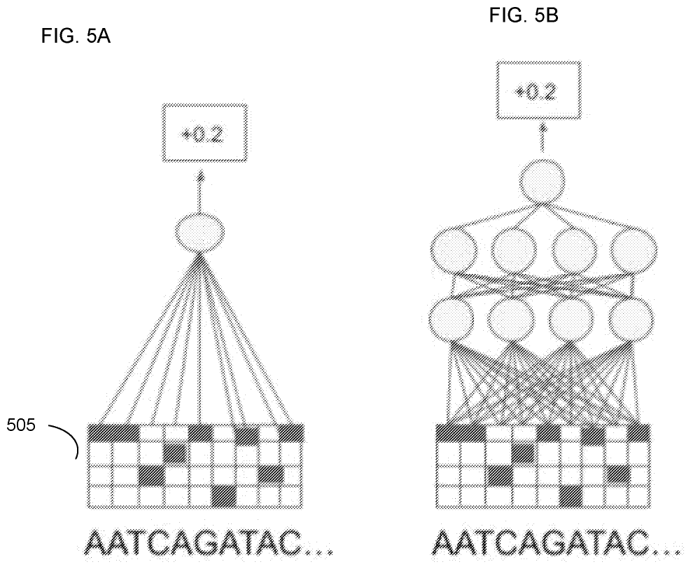

FIGS. 5A-5B shows schematic illustrations of the linear regression and neural networks models using the independent site (IS) representation for the input. FIG. 5A shows the linear regression model with the IS representation 405. The one-hot encoded inputs are weighted according to the linear regression parameters to provide the predicted fitness value. FIG. 5B shows the NN model with the IS representation being multiplied by the weights of the nodes and providing the predicted fitness value. In the IS representation, each column corresponds to a different site. A column can be referred to as an independent site vector. A row corresponds to a different residue (e.g., a particular nucleotide or amino acid). A black square indicates that a particular residue is at the site. Only one element can be black for any given column (independent site vector). Accordingly, encoding the viral vector sequence as the feature set can comprises one-hot encoding each residue of the viral vector sequence to produce a plurality of independent site vectors.

### 2. Experimental Validation of the Predictive Models.

Before proceeding to using our predictive model for library design, we first validated the “NN, 100” model by identifying and synthesizing five individual 7-mer insertion sequences that were not present in our original experiment dataset. These five sequences were chosen to span a broad range of predicted log enrichment scores (−5.84 to 4.83). The five variants were packaged individually into viruses, harvested, and titered by quantifying the resulting number of genome-containing particles using digital-droplet PCR, e.g., as described herein.

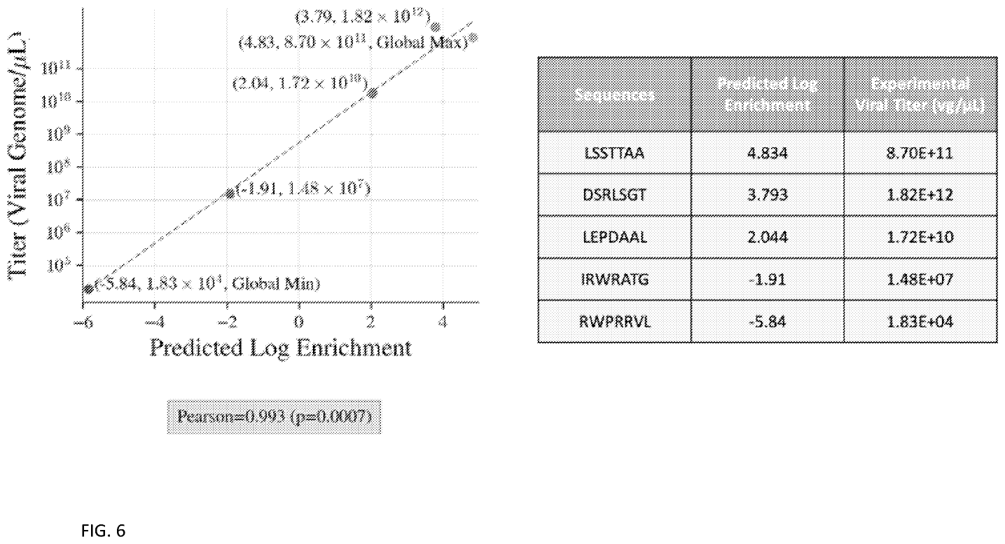

FIG. 6 shows experimental titers (vg/μL) versus predicted log enrichment scores for five variants selected to span a broad range of predicted log enrichment scores. Log enrichment scales are computed using natural logarithm. FIG. 6 shows good relative correspondence of the predicted enrichment with the measured viral titers, and thus the relative ability to package between sequences is captured, which allows selection of viral vectors that are more likely to package. High titer values indicated the variant was capable of packaging its genome properly in the assembled capsid. The agreement between model predictions and corresponding experimental measurement of vector titers (1.83×10⁴ to 8.70×10¹¹ viral genomes (vg)/μL) demonstrates that the predictive model was sufficiently accurate to be used for library design.

### 3. Example Techniques for Model Training and Evaluation

Various techniques can be used for model training and evaluation. For example, many different optimization algorithms can be used (e.g., stochastic gradient descent, Adam, etc.), and regularization techniques (L2, Lasso, etc.) that could be used to train these models. One example is as follows.

The data processing can yield a data set of the form {(xᵢ,yᵢ,σᵢ²)}ᵢ₌₁^{M} where the xᵢ are unique insertion sequences, yᵢ are log enrichment scores associated with the insertion sequences, σᵢ² are the estimated variances of the log enrichment scores, and M=8,555,729 is the number of unique insertion sequences in the data. In one implementation, we randomly split this data set into a training set containing 80% of the data and a test set containing the remaining 20% of the data. Other percentage splits can be used.

The distribution of a log enrichment score given the associated insertion sequence can be assumed to be

$${y}_{i} \vert {x}_{i} , {σ}_{i}^{2} ∼ N ( {f}_{θ} ( {x}_{i} ) , {σ}_{i}^{2} )$$

where f_{θ} is a function with parameters θ that parameterizes the mean of the distribution and represents a predictive model for log enrichment scores. We determined suitable settings of the parameters θ with Maximum Likelihood Estimation (MLE). The log-likelihood of the parameters of this model given the training set of M′≤M data points is given by

$$ℓ ( θ ; {\{ {x}_{i} , {y}_{i} , {σ}_{i}^{2} \}}_{i = 1}^{{M}^{′}} ) = \frac{{M}^{′}}{2} log 2 π - \frac{1}{2} {\sum }_{i = 1}^{{M}^{′}} [ log {σ}_{i}^{2} + \frac{1}{{σ}_{i}^{2}} {( {y}_{i} - {f}_{θ} ( {x}_{i} ) )}^{2} ]$$

Performing MLE by optimizing this likelihood with respect to the model parameters, θ, results in the weighted least-squares loss function in Equation 3.

$$L ( θ ) = {\sum }_{i = 1}^{M} \frac{1}{{σ}_{i}^{2}} {( {y}_{i} - {f}_{θ} ( {x}_{i} ) )}^{2} , \qquad ( Eqn . 3 )$$

For the linear forms of f_{θ}, the loss (Equation 3) is a convex function which can be solved exactly for the minimizing ML parameters. In order to stabilize training, we used a small amount of l₂ regularization for the Neighbors and Pairwise representations (with regularization coefficients 0.001 and 0.0025, respectively, chosen by cross-validation). For the neural network forms of f_{θ}, the objective (Equation 3) is non-convex, and we use stochastic optimization techniques to solve for suitable parameters. These models can be implemented using various software packages, such as in TensorFlow [37]. The built-in implementation of the Adam algorithm [38] can be used to approximately solve Equation 3.

To assess the prediction quality of each model, we calculated the Pearson correlation between the model predictions and observed log enrichment scores for different subsets of the sequences in the test set. Our aim is to use these models to design a library of sequences that package well (i.e., would be highly enriched in the post-selection library). We, therefore, assessed how well the models perform for highly enriched sequences by progressively culling the test set to only include sequences with the largest observed log enrichment scores (FIGS. 4A-4B).

### C. Optimizing Library

Once trained, individual sequences and set of sequences (libraries) can be evaluated amongst each other to determine which ones have better packaging fitness. Library 238 is an example that can be evaluated and can be defined as a nucleotide sequence or an amino acid sequence. A library distribution 230 can be used to generate library 238. Library distribution 230 can defined for a length of a nucleotide sequence that is inserted into the viral vector sequence. As mentioned above, four probabilities (one for each nucleotide) can exist for each position in the inserted sequence. Thus, each position has a probability distribution (e.g., 20% A, 30% C, 35% G, 15% T), and the entire inserted sequence can include a set of probability distributions. The term probability distribution can also refer to the probabilities for the entire inserted sequence. For a 21-nt sequence, the entire probability distribution has 84 probabilities. At 236, library distribution 230 can be randomly samples to obtain library 238.

Each sequence from library 238 can be input to predictive model 220, which can provide a set of predicted fitness values 222. A library fitness value 224 can be determined from the individual fitness values. For example, an average can be taken of the individual fitness values.

In addition to library fitness value 224, a diversity score can be used. As shown in FIG. 2, a statistical entropy 232 can be determined as a diversity score for library distribution 230. The formula (sum of P*log (P)) for statistical entropy 232 is shown on the line from library distribution 230 to balance objectives 226 (e.g., a weighted sum of the fitness and diversity terms/scores), illustrating that diversity can also be taken into account for the overall comparison of libraries.

Depending on the value for balance objectives 226, library distribution 230 can be updated, e.g., by changing any one or more of the probabilities. A gradient can be determined from previous evaluations of previous library distributions, and the gradient can be used to select new probabilities in the library distribution to minimize balance objectives 226. The new library distribution can then be sampled to obtain a new library that can be evaluated to determine a new objective score for the new library, and the optimization can proceed until convergence (e.g., a specified number of iterations, a loss value below a threshold, or the change in the loss for a specified number of iterations is below a threshold).

### D. Flowchart for Training Prediction Model

As described above, a machine learning model can be trained to predict a fitness value of a viral vector, which includes an insertion sequence, to provide a physical property/characteristic, such as packaging.

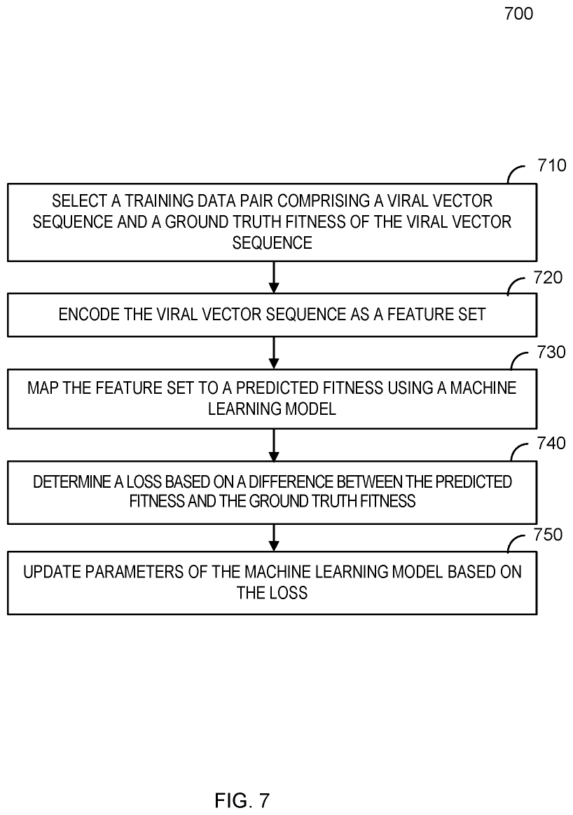

FIG. 7 is a flowchart illustrating a method for training a machine learning model to predict fitness (e.g., packaging fitness) of viral vector sequences. Method 700 may be executed by one or more of the systems described herein. In some embodiments, method 700 may be implemented by computing system 100 shown in FIG. 1. In some embodiments, method 700 may be implemented by machine learning module 108, stored in non-transitory memory 106.

It will be appreciated that method 700, as well as the other methods disclosed herein, are compatible with various architectures of machine learning models. In some embodiments, a machine learning model may comprise a neural network, or deep neural network. In some embodiments, a machine learning model may comprise a linear model. For example, a fitness value (e.g., packaging fitness) can be determined based on a linear combination of input features extracted from the viral vector sequence. A neural network architecture may comprise an input layer, one or more hidden layers, and an output layer. In one example, a neural network may comprise two densely connected hidden layers with tanh activation functions. In one example, a number of nodes in the two hidden layers may comprise between 100 and 1000 nodes in each hidden layer. It will be appreciated that the above machine learning model architectures are exemplary, and other model architectures are encompassed by the current disclosure.

At operation 710, a training data pair is selected from a plurality of training data pairs. In some embodiments, a training data pair comprises a viral vector sequence (including an insertion sequence) and a corresponding ground truth fitness (e.g., a packaging fitness such as an enrichment score as described herein). The ground truth fitness value is an experimentally measured property of the viral vector sequence to deliver a gene therapy to a cell. A packaging fitness is such an example. Thus, the ground truth fitness value can be a ground truth packaging fitness value. Method 700 can be performed many times for many training data pairs (e.g., each one in a library). As examples, a library can include at least 1,000, 5,000, 10,000, 50,000, or 100,000 sequences.

A packaging fitness can be a measured indicator of the probability of viral vector proteins, encoded by the viral vector sequence, to successfully package as a viral vector particle. The ground truth packaging fitness value can be a function of a first abundance of the viral vector sequence, measured before a packaging process, and a second abundance of the viral vector sequence, measured after the packaging process. For example, the ground truth packaging fitness may be estimated according to the below equation:

$${y}_{i} = log \frac{{n}_{i}^{p o s t}}{{n}_{i}^{p r e}} - log \frac{{N}^{p o s t}}{{N}^{p r e}} ,$$

as described above.

The training data pair may be intelligently selected by the computing system based on one or more pieces of metadata associated with the training data pair. In some embodiments, the computing system may select a training data pair from training data 112 based on a type of viral vector sequence of the training data pair. As an example, a machine learning model can be trained to predict fitness for adeno associated viruses (AAVs), and the computing system may select a training data pair comprising an AAV sequence and a corresponding ground truth fitness. In other embodiments, the training data pair may be acquired via communicative coupling between the computing system and an external storage device, such as via Internet connection to a remote server.

At operation 720, the computing system encodes the viral vector sequence of the training data pair as a feature set. The encoding may be done in various ways, e.g., as described herein. In some embodiments, the viral vector sequence may be encoded in an “Independent Site” (IS) representation, wherein amino acids in the viral vector sequence are one-hot encoded. In some embodiments, the viral vector sequence may be encoded in a “Neighbors” representation, wherein interactions between neighboring amino acid positions in the viral vector sequence are one-hot encoded. In some embodiments, the viral vector sequence may be encoded in a “Pairwise” representation, wherein all possible interactions between residue positions of the viral vector sequence are one-hot encoded. The feature set produced at operation 304 may comprise one or more, or each of the “independent site” representation, the “neighbors” representation, and the “pairwise” representation. The encoding for the “neighbors” and “pairwise” representation can comprise encoding each pair of adjacent (neighboring) residues of the viral vector sequence as a vector to produce a plurality of interaction feature vectors.

At operation 730, the computing system maps the feature set to a predicted fitness using a machine learning model. The predicted fitness value can be a predicted packaging fitness value or fitness value for a different characteristic. In embodiments where the machine learning model is a linear model, each feature of the feature set may be weighted according to an associated weight/parameter of the linear model and combined to produce the predicted fitness of the viral vector sequence. In embodiments wherein the machine learning model comprises a neural network, the feature set may be fed to an input layer of the neural network, and propagated through one or more hidden layers, wherein output from the one or more layers is fed to an output layer. The output layer of the neural network may produce the predicted fitness of the viral vector based on the input received from a last hidden layer.

At operation 740, the computing system may calculate a loss for the machine learning model based on a difference between the predicted fitness and the ground truth fitness. In one example, the loss may be given by the following equation:

$${\sum }_{i = 1}^{M} \frac{1}{{σ}_{i}^{2}} {( {y}_{i} - {f}_{θ} ( {x}_{i} ) )}^{2}$$

where M is a total number of training data pairs, yᵢ is the ground truth fitness of viral vector sequence i, f_{θ}(xᵢ) is the predicted fitness of viral vector sequence i, and σᵢ² is the variance (used as the weight) associated with the ground truth fitness yᵢ. Other examples do not include the variance.

When a packaging fitness is determined, the variance can be determined as an estimate based on the number of pre-packaging or post-packaging sequences. As can be seen, the variance acts to downweight the loss in proportion to the magnitude of the variance, thereby de-emphasizing training data pairs which may be highly variable, allowing the machine learning model to prioritize training data pairs with lower variance. In one example, the variance, a may be determined according to the following equation (described above):

$$\frac{1}{{n}_{i}^{p o s t}} ( 1 - \frac{{n}_{i}^{p o s t}}{{N}^{post}} ) + \frac{1}{{n}_{i}^{p r e}} ( 1 - \frac{{n}_{i}^{p r e}}{{N}^{pre}} ) .$$

The variance can also used for weighting when properties other than packaging are optimized.

In some embodiments, an unweighted loss may be used, wherein the variance term in the above equation may be set to a constant value of 1 for all training data pairs, thereby giving an equal weight/importance to each training data pair.

At operation 750, the parameters of the machine learning model are adjusted based on the loss. The loss may be back propagated through the layers of the machine learning model to update the parameters (e.g., weights and biases) of the machine learning model. In some embodiments, back propagation of the loss may occur according to a gradient descent algorithm, wherein a gradient of the loss function (a first derivative, or approximation of the first derivative) is determined for each weight and bias of the machine learning model. Each weight (and bias) of the machine learning model is then updated by adding the negative of the product of the gradient determined (or approximated) for the weight (or bias) and a predetermined step size, according to the below equation:

$${P}_{i + 1} = {P}_{i} - Step \frac{\partial ( loss )}{\partial {P}_{i}}$$

where Pᵢ₊₁ is the updated parameter value, Pᵢ is the previous parameter value, Step is the step size, and

$$\frac{\partial ( loss )}{\partial {P}_{i}}$$

is the partial derivative of the loss with respect to the previous parameter. In some embodiments, a gradient descent algorithm, such as stochastic gradient descent, may be used to update parameters of the machine learning model to iteratively decrease the loss.

Method 700 may be repeated for many training data pairs for many iterations, e.g., until the parameters of the machine learning model converge, an accuracy is obtained (for the training data or for a separate validation dataset), or the rate of change of the parameters of the machine learning model for each iteration of method 300 are less than a threshold rate of change. In this way, method 700 enables a machine learning model to be trained to infer fitness for viral vector sequences. Example number of training data pairs can be at least 1,000, 5,000, 10,000, 50,000, or 100,000 training data pairs.

### IV. Model-Guided Library Design with Diversity

Having validated the ability of the prediction model to determine relative packaging fitness among different viral vectors, the prediction model can be used to determine a library of viral vector sequences with improved packaging. For example, an average packaging fitness (e.g., library fitness value 224) for each of a set of libraries can be used to determine an optimal library from the set. Some embodiments can be used to design a library that packages better than other libraries (e.g., the NNK library), while maintaining good diversity of different types of sequences in the library. Such a library design can also be used for other types of fitness values, examples of which are provided herein.

Inherent to the challenge of having high diversity and high library fitness values is a trade-off between library diversity and mean predicted packaging fitness of the library. For example, the mean predicted packaging fitness is maximized with a library that contains only a single variant with the highest predicted fitness, while diversity is maximized with a library uniformly distributed across sequence space, irrespective of packaging fitness. The library that is most effective for downstream selections will lie between these two extremes, balancing mean packaging fitness with diversity.

As described for FIG. 2, the trained model can be used to determine the packaging fitness for an entire library based on the sequences (or a random sampling of the sequences defined by probability distributions). An objective score can be determined for each library, where the objective score includes the fitness and a diversity of the library. A library can then be updated to increase the objective score, e.g., by selecting a different set of sequences, which can be accomplished by sampling different probability distributions. Various optimal libraries can be generated for different tradeoff coefficients (A).

A library can be defined as a sequence set (set of individual sequences) or a distribution set (set of probabilities from which individual sequences can be sampled). The packaging fitness for a sequence set can be determined using the prediction model to determine the predicted fitness value of each sequence of the sequence set. A fitness score can be an average or other statistical value (e.g., mode or median) of the individual fitness values. The packaging fitness for a distribution set can be determined by randomly sampling the distribution set to generate a sequence set, and then using the model to determine the predicted fitness score of each sequence of the sequence set. A weighted average can be used, where each fitness value is weighted by the likelihood of that sequence being generated for the given distribution set.

Diversity can be defined for a distribution set using the probabilities. For example, diversity (e.g., entropy) can correspond to −Σᵢ₌₁^{N} P(xᵢ) log P(xᵢ), where P(xᵢ) is a probability of occurrence of viral vector sequence i in the viral vector library. An equal probability for each type of residue at each position provides the highest diversity score. Diversity for a sequence set can be determined in various ways. For example, the computer system can determining the probability distribution of residues at each position (effectively determining a distribution set), and then determine the diversity as described above. As another example, one can estimate the entropy of the unconstrained distribution via sampling techniques if the sequences are sampled from this distribution. If the sequences are sampled from an unknown distribution, one can fit a probability distribution (e.g., a Potts model) to the sequences and use the entropy of that distribution as the estimate.

A distribution set can be optimized by updating the probabilities, e.g., the probability of each residue (e.g., nucleotide or amino acid) at each position in in the sequence. For a nucleotide sequence of length N positions, the number of probabilities would be 4×N. For a peptide sequence of length K positions (e.g., N/3), the number of probabilities would be 20×K. The probabilities of the viral vector library can be updated to increase the objective score, which can be a weighted sum of the fitness and diversity terms. A tradeoff coefficient A can be defined as a ratio of the weights for the fitness and diversity terms. Such a ratio provides a relative weight between fitness and diversity.

A Pareto frontier can be determined for different tradeoff coefficients A. In this manner, a library designer can select a distribution set corresponding to a desired tradeoff. By accounting for diversity, a diversity-constrained optimal library design is employed.

An unconstrained library (unconstrained sequence set) can be generated by selecting individual sequences, i.e., not according to any probability distributions. The cost to physically generate an unconstrained library is higher. The fitness score can be generated as an average of the individual fitness values, just like a constrained library. The diversity of an unconstrained sequence set can be determined, as described above, e.g., by determining the distribution of residues at each position. Unconstrained libraries provide more control over the contents of the library than constrained libraries but are substantially more expensive per oligonucleotide (each of which must be synthesized). Therefore, in considering constrained vs unconstrained libraries, one is trading off control for library size.

### A. Constrained Library

Because the best trade-off between the two extremes (a single variant with highest fitness and a library uniformly distributed across sequence space) is not clear a priori, some embodiments can provide library design tools to trace out an optimal trade-off curve, also known as a Pareto frontier.

### 1. Optimal (Pareto) Frontier

Each point lying on this optimal frontier represents a library for which it is not possible to improve one desiderata (packaging or diversity), without hurting the other. Such a Pareto optimal frontier, therefore, allows assessment of what mean library packaging fitness can be achieved for any given level of diversity. To generate each point (library) that lies on the optimal frontier curve, we define a library optimization objective (objective score) that seeks to maximize mean predicted fitness subject to a library diversity constraint controlled by the tradeoff value λ. This knob, λ, controls the tradeoff between library diversity and packaging ability; we set λ to different values to trace out the Pareto frontier.

We quantified the diversity of each theoretical library by computing the statistical entropy (sum of P*log (P))) of the probabilistic distribution it corresponds to. We refer to this overall methodology enabling tracing out the optimal curve as diversity-constrained optimal library design. We note that the optimization problem is challenging to solve exactly (i.e., it is non-convex). Consequently, libraries computed as we trace out X may not lie exactly on the optimal frontier. However, the frontier can nevertheless be inferred approximately, providing useful insights. We applied this diversity-constrained optimal library design methodology to the design of an improved AAV5-7mer peptide insertion library, yielding some striking implications.

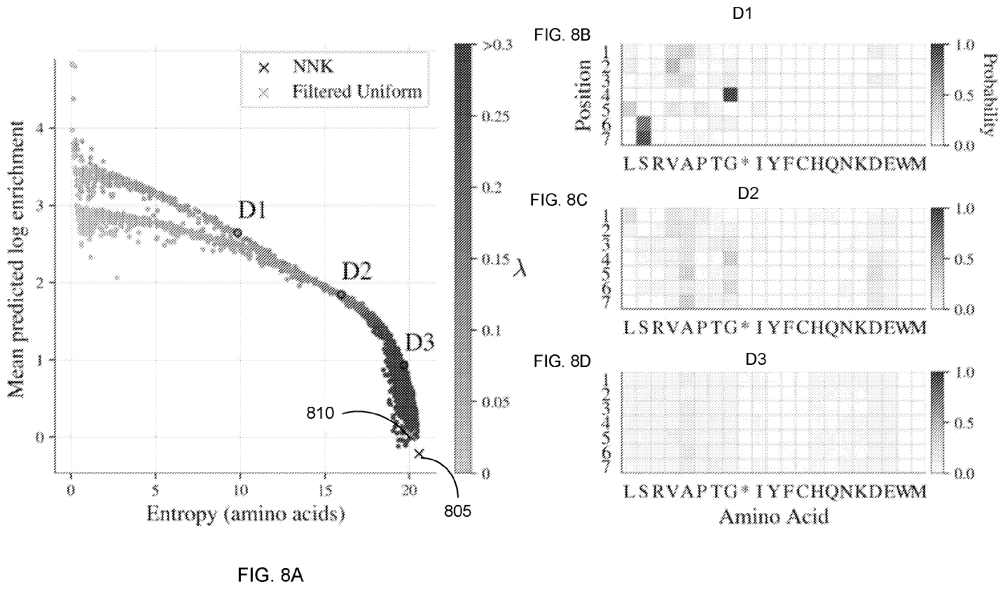

FIGS. 8A-8D show results for designed AAV5-based 7-mer insertion libraries, including a Pareto frontier (FIG. 8A) and probabilities for different distribution sets (FIGS. 8B-8D). Each point in FIG. 8A represents a theoretical library designed with our diversity-constrained optimal library approach, with one particular diversity constraint, A (higher values yields more diverse libraries). Entropy indicates diversity of the library distribution, while mean predicted log enrichment indicates overall library fitness; both quantities can be computed from the theoretical library distribution, e.g., as described above.

The baseline NNK library 805 is denoted with a black “x”. Three designed libraries (representative of three important areas of the curve) have been circled and labeled D1-D3 for reference. Due to the non-convex optimization problem, some dots are suboptimal (i.e., lie strictly below or to the left of other dots) and are therefore further from the optimal frontier, but are displayed for completeness.

Remarkably, the NNK library has a dramatically poor mean predicted log enrichment (MPLE), much lower than any designed library. In contrast, library D3 had nearly identical diversity but substantially higher mean packaging fitness (top 50% of all designed libraries). This observation implies that D3 effectively dominates NNK in the sense that we increased the predicted packaging fitness without taking much loss to the diversity. Such concrete conclusions can be drawn from a Pareto frontier whenever one point on the frontier lies vertically above another. In addition, we see that compared to D3, D2 is less diverse but is predicted to package better (2.0-fold higher MPLE). Similarly, D1 is less diverse than D2, but again is predicted to package better (1.4-fold higher MPLE).

Although the original motivation for creating the NNK library was to reduce the number of stop codons, it does not eliminate them entirely. Therefore, for further comparison, we computed the mean packaging fitness and diversity of the theoretical library containing all possible sequences, except for any containing a stop codon. In practice, such a library is not physically realizable using this position-wide nucleotide specification strategy but serves as a useful comparator. We call this the “filtered uniform” library; the cyan “x” denotes the “filtered uniform” library 810 that is uniform over all 21-mer nucleotide sequences except for those containing stop codons. We find that, on the one hand, it does have slightly higher mean packaging fitness than NKK, and correspondingly less diversity. However, these differences are negligible compared to the differences between NNK and D3, suggesting that further removal of stop codons is not the primary mechanism by which our ML-designed libraries achieve higher predicted packaging fitness.

FIGS. 8B-8D show designed library parameters (probability of each amino acid at each position) for the three designed libraries D1-D3, respectively, highlighted in FIG. 8A. In this example, the distribution set is defined by the probability of the amino acids in the 7-mer sequence. As one can see, the probabilities of some amino acids are higher for D1, which has lower diversity. The probabilities for D2 are somewhat lower, and the probabilities for D3 are almost uniform, giving it the highest diversity.

### 2. Experimental Validation of Designed Libraries

We synthesized two designed libraries (D2 and D3) from our optimality curve (FIG. 8A) to assess the accuracy of the designed libraries' trade-off between diversity and mean packaging fitness. Later we also tested D2 in a downstream selection task of infecting brain tissue. The library D2 was chosen for being at the “elbow” of the curve, suggestive of a library making a good trade-off. The library D3 was chosen because, as discussed earlier, it dominates NKK by achieving much higher predicted packaging fitness with a negligible drop in diversity.

After experimentally constructing and deep sequencing these two designed libraries, we first checked that the physically realized library matched the statistics of the theoretical designed library distribution. Indeed, we found that the empirically observed position-wise probabilities for each amino acid in each of the designed libraries was within 5% of the designed specification (Tables 1-2). Table 1 provides the nucleotide probabilities for the 21-bp sequence for the designed libraries D2 and D3. Table 2 provides the probability distribution of the actual sequences in the synthesized libraries generated based on D2 and D3 respectively.

TABLE 1
Marginal probabilities of library D2 and
D3 nucleotides at 21-bp position chart.

| Li- | | | | | Li- | | | | |
|---|---|---|---|---|---|---|---|---|---|
| brary | | | | | brary | | | | |
| D2 | A | T | C | G | D3 | A | T | C | G |
| 1 | 0.12 | 0.04 | 0.39 | 0.45 | | 0.21 | 0.09 | 0.43 | 0.27 |
| 2 | 0.18 | 0.47 | 0.3 | 0.05 | | 0.22 | 0.25 | 0.37 | 0.16 |
| 3 | 0.21 | 0.19 | 0.28 | 0.32 | | 0.25 | 0.28 | 0.27 | 0.2 |
| 4 | 0.14 | 0.02 | 0.19 | 0.65 | | 0.27 | 0.12 | 0.13 | 0.48 |
| 5 | 0.23 | 0.33 | 0.29 | 0.15 | | 0.2 | 0.35 | 0.27 | 0.18 |
| 6 | 0.28 | 0.24 | 0.25 | 0.23 | | 0.22 | 0.23 | 0.22 | 0.33 |
| 7 | 0.35 | 0 | 0.14 | 0.51 | | 0.22 | 0.08 | 0.16 | 0.54 |
| 8 | 0.13 | 0.17 | 0.36 | 0.34 | | 0.32 | 0.19 | 0.34 | 0.15 |
| 9 | 0.21 | 0.31 | 0.31 | 0.17 | | 0.25 | 0.17 | 0.27 | 0.31 |
| 10 | 0.13 | 0 | 0.06 | 0.81 | | 0.24 | 0.07 | 0.43 | 0.26 |
| 11 | 0.26 | 0.12 | 0.22 | 0.4 | | 0.34 | 0.35 | 0.2 | 0.11 |
| 12 | 0.16 | 0.29 | 0.36 | 0.19 | | 0.22 | 0.26 | 0.28 | 0.24 |
| 13 | 0.09 | 0 | 0.08 | 0.83 | | 0.28 | 0.06 | 0.17 | 0.49 |
| 14 | 0.36 | 0.12 | 0.37 | 0.15 | | 0.45 | 0.14 | 0.29 | 0.12 |
| 15 | 0.13 | 0.49 | 0.24 | 0.14 | | 0.2 | 0.27 | 0.3 | 0.23 |
| 16 | 0.22 | 0 | 0.13 | 0.65 | | 0.32 | 0.08 | 0.19 | 0.41 |
| 17 | 0.29 | 0.08 | 0.24 | 0.39 | | 0.23 | 0.2 | 0.35 | 0.22 |
| 18 | 0.1 | 0.42 | 0.34 | 0.14 | | 0.23 | 0.27 | 0.36 | 0.14 |
| 19 | 0.16 | 0.01 | 0.09 | 0.74 | | 0.2 | 0.04 | 0.32 | 0.44 |
| 20 | 0.28 | 0.11 | 0.47 | 0.14 | | 0.39 | 0.17 | 0.3 | 0.14 |
| 21 | 0.17 | 0.35 | 0.3 | 0.18 | | 0.26 | 0.25 | 0.24 | 0.25 |

TABLE 2
Synthesized marginal probabilities of library D2 and D3.

| Li- | | | | | Li- | | | | |
|---|---|---|---|---|---|---|---|---|---|
| brary | | | | | brary | | | | |
| D2 | A | T | C | G | D3 | A | T | C | G |
| 1 | 0.13 | 0.05 | 0.40 | 0.42 | | 0.22 | 0.10 | 0.42 | 0.25 |
| 2 | 0.18 | 0.50 | 0.27 | 0.05 | | 0.22 | 0.29 | 0.34 | 0.15 |
| 3 | 0.20 | 0.21 | 0.27 | 0.32 | | 0.24 | 0.32 | 0.25 | 0.19 |
| 4 | 0.14 | 0.03 | 0.18 | 0.65 | | 0.26 | 0.14 | 0.12 | 0.48 |
| 5 | 0.22 | 0.36 | 0.28 | 0.14 | | 0.19 | 0.39 | 0.25 | 0.17 |
| 6 | 0.27 | 0.27 | 0.24 | 0.22 | | 0.21 | 0.26 | 0.21 | 0.32 |
| 7 | 0.35 | 0 | 0.14 | 0.51 | | 0.22 | 0.09 | 0.15 | 0.54 |
| 8 | 0.13 | 0.19 | 0.36 | 0.32 | | 0.32 | 0.22 | 0.32 | 0.14 |
| 9 | 0.20 | 0.34 | 0.30 | 0.16 | | 0.24 | 0.20 | 0.25 | 0.31 |
| 10 | 0.13 | 0 | 0.06 | 0.81 | | 0.25 | 0.08 | 0.41 | 0.26 |
| 11 | 0.27 | 0.14 | 0.21 | 0.38 | | 0.33 | 0.38 | 0.18 | 0.11 |
| 12 | 0.16 | 0.31 | 0.35 | 0.18 | | 0.21 | 0.29 | 0.26 | 0.24 |
| 13 | 0.09 | 0 | 0.08 | 0.83 | | 0.28 | 0.07 | 0.16 | 0.49 |
| 14 | 0.37 | 0.14 | 0.35 | 0.14 | | 0.45 | 0.16 | 0.27 | 0.12 |
| 15 | 0.12 | 0.53 | 0.22 | 0.13 | | 0.19 | 0.31 | 0.28 | 0.22 |
| 16 | 0.22 | 0 | 0.12 | 0.66 | | 0.32 | 0.09 | 0.18 | 0.41 |
| 17 | 0.30 | 0.10 | 0.24 | 0.36 | | 0.23 | 0.24 | 0.33 | 0.20 |
| 18 | 0.1 | 0.45 | 0.33 | 0.14 | | 0.22 | 0.30 | 0.35 | 0.13 |
| 19 | 0.16 | 0.01 | 0.09 | 0.74 | | 0.2 | 0.04 | 0.32 | 0.44 |
| 20 | 0.29 | 0.14 | 0.44 | 0.14 | | 0.38 | 0.19 | 0.29 | 0.14 |
| 21 | 0.16 | 0.41 | 0.28 | 0.18 | | 0.25 | 0.30 | 0.23 | 0.22 |

Total reads assessed by deep sequencing: 193228 (library D2) and 212388 (library D3)

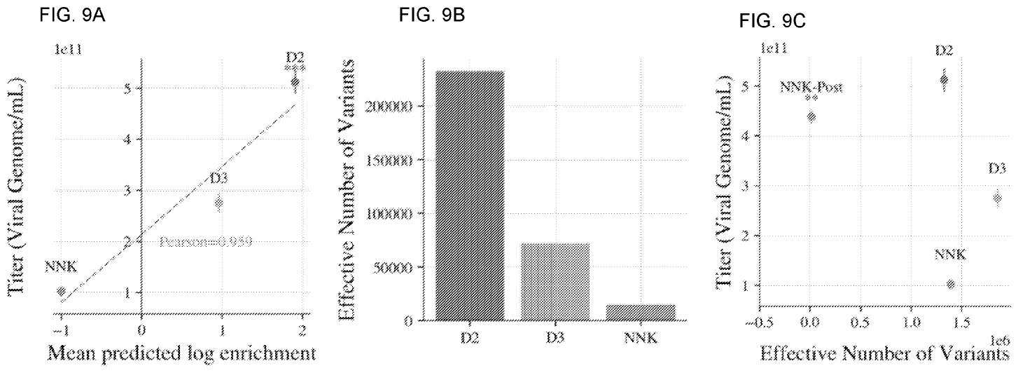

Having validated that the constructed libraries were as specified, we packaged and harvested each library using the same methods as for the NNK library, yielding a pre- and post-packaged version of each. Next, we assessed to what degree the MPLE of each library reflected the measured library titers and found a strong positive Pearson correlation between them (r=0.959, FIG. 9A).

FIGS. 9A-9C shows a comparison of ML-designed libraries D2 and D3 to the NNK library. FIG. 9A shows experimental titers (viral genome/mL), after packaging, plotted against the MPLE for the different libraries. The correlation shows that the prediction model accurately provides relative packaging ability of each library.

As discussed earlier, D3 dominates the NNK library in fitness (one lies vertically above the other) and is thus predicted to be the better library. The choice between D3 and D2 is less clear, as they trade off packaging fitness and diversity. To assess such tradeoffs, we subjected each of D2, D3 and NNK to one round of packaging selection (e.g., seed cells, transfect, incubate for a couple days, lyse cells, purify virus), and then estimated the effective number of variants remaining from the deep sequencing data. A larger effective number of variants after selection suggests that a library contains more variants able to package.

FIG. 9B shows a comparison of the effective number of variants present in each library after packaging. Such an analysis revealed D2 to be better than D3, which is consistent with a higher enrichment. Looking back at our measured titers, designed library D2 (MPLE˜2.0) showed a 5-fold higher packaging titer than that of the NNK library (MPLE˜−0.9) with titers of 5.12×10¹¹ and 1.02×10¹¹ vg/mL, respectively.

FIG. 9C shows experimental titers and effective number of variants for D2, D3, NNK, and NNK-post libraries. The NNK-post library represents the NNK library after an additional round of packaging selection. One-way ANOVA followed by Tukey test (** p<0.01 compared to D2, * * * p<0.001 compared to NNK). In all cases, experimental titers are measured on 3 replicates. Graphs show means+/−SD.

The packaging titer (4.38×10¹¹ vg/mL) of the NNK library after another round of packaging selection (NNK-post), finding that its titer was lower than that of D2 (5.12×10¹¹ vg/mL). This result suggests that the additional round of packaging was not enough to lift the NNK library's titer level to that of library D2. Note, also, that the NNK-post library has only 1.48×10⁴ effective variants compared to the 1.33×10⁶ effective variants in D2. Collectively, these experimental results suggest that our ML-guided library design procedure yielded a more useful library than the NNK library, the peptide insertion library of choice for AAV directed evolution experiments.

### 3. Diversity-Constrained Optimal Library Design

An example technique to determine the optimal libraries on the Pareto frontier is described in this section. We developed a general framework for sequence library design that (i) can be used with any predictive model of fitness, (ii) is broadly applicable to different library construction mechanisms (e.g., error prone PCR, site-specific marginal probability specification, individual synthesized sequences), and (iii) is simple to implement and extend. This framework balances mean predicted packaging fitness with entropy, a measure of diversity for probability distributions which has been used extensively in ecology to describe the diversity of populations [39]. This example approach is based on a maximum entropy formalism: we represent libraries as probability distributions and aim to find maximum entropy distributions that maximize entropy while also satisfying a constraint on the mean fitness, which is predicted by a user-specific model such as a neural network.

Let χ be the space of all sequences that may be included in a library (e.g., all amino acid sequences of length 7). We consider a library to be an abstract quantity represented by a probability distribution with support on χ. Let [symbol] represent all such libraries and pϵ [symbol] one particular library. The entropy of this library is given by [40]:

$$H [ p ] = - {\sum }_{x ∈ χ} p ( x ) log p ( x )$$

Now, let f(x) be a predictive model of fitness (e.g., from a trained neural network). Our goal is to find a diverse library, p, where the mean predicted fitness in the library, [symbol] ₚ₍ₓ₎[f(x)], is as high as possible. Formally, we want to find the library with the largest entropy such that the mean predicted fitness is above some cutoff. This objective is written

$${max}_{p ∈ ℘} H [ p ]$$

$$s . t . {𝔼}_{p ( x )} [ f ( x ) ] \geq a$$

where a is the cutoff on the mean predicted fitness. The solution to this optimization problem is given by [41]:

$${p}_{λ} ( x ) = \frac{1}{Z ( λ )} exp ( \frac{f ( x )}{λ} ) \qquad ( Eqn . 4 )$$

where λ>0 is a Lagrange multiplier that is a monotonic function of the cutoff a and Z(λ)=Σ_{xϵχ} exp (f(x)/λ) is a normalizing constant.

Equation 4 gives the probability mass of what is known as the maximum entropy distribution. The parameter λ controls the balance between diversity and mean fitness in the library (higher λ corresponds to more diversity). Each library, p_{λ}, represents a point on a Pareto optimal frontier of libraries, which balances diversity and mean predicted fitness; these distributions cannot be perturbed in such a manner as to both increase the entropy and the mean fitness. Theoretically, the entire Pareto frontier could be traced out by calculating the mean predicted fitness and entropy of p_{λ} for every possible setting of λ. In this example, we pick a discrete set of λ that traces out a practically useful curve.

As written so far, this framework can be used to select a particular library distribution, p_{λ}(x), with value λ, from the Pareto optimal curve. Then, if designing libraries comprised of individually specified sequences, one can sample individual sequences from this distribution, thereby designing a realizable, synthesizable library. However, for many cases of practical interest, it is not cost-effective to synthesize individual sequences. Some implementations can consider a more affordable library construction mechanism: a library of oligonucleotides is generated in a stochastic manner based on specified position-wise nucleotide probabilities. Because this position-wise nucleotide specification strategy does not allow one to specify individual sequences, we refer to libraries constructed in this way as constrained. In the next section, we describe how we use our design framework to set the parameters of these constrained libraries.

### 4. Maximum Entropy Design for Constrained Libraries

In this section, we describe the design of libraries that are not specified at the level of individual sequences, but rather at the (less precise) level of position-specific distributions. In particular, we controlled the marginal probability of each nucleotide at each position. The probability mass function of the distribution representing a library specified by position-wise probabilities is given by:

$${q}_{ϕ} ( x ) = {\prod }_{j = 1}^{L} {\sum }_{k = 1}^{K} {q}_{{ϕ}_{j}} ( {x}^{j} = k ) {δ}_{k} ( {x}^{j} )$$

where L is the sequence length, K is the alphabet size (i.e., K=4 for nucleotide libraries), ϕϵ [symbol] ^{L×K} is a matrix of distribution parameters, ϕⱼ is the j^{th} row of ϕ, δₖ(x^{j})=1 if x^{j}=k and zero otherwise, and

q_{ϕ}_{ⱼ}(x^{j}=k)=e^{ϕ}^{ⱼₖ}/Σᵢ₌₁^{K}e^{ϕ}^{ⱼₗ} (Eqn. 5).

For an arbitrary predictive model (such as a neural network to predict log enrichment scores from sequence), the maximum entropy distribution (Equation 4) will generally not have the form of Equation 5. To apply the maximum entropy formulation to the design of libraries that are constrained to take a particular form, what we refer to as constrained library design, we take a variational approach: for a single, fixed value of λ, we find the constrained library distribution, q_{θ}, that is the best approximation to the maximum entropy library distribution, p_{λ} in terms of the KL divergence,

$${ϕ}_{λ} = arg {min}_{ϕ} {D}_{K L} [ {q}_{ϕ} ‖ {p}_{λ} ] = arg {max}_{ϕ} {𝔼}_{{q}_{ϕ} ( x )} [ f ( x ) ] + λ H [ {q}_{ϕ} ] . \qquad ( Eqn . 6 )$$

Our objective (Equation 6) is a non-convex function of the library parameters. The Stochastic Gradient Descent (SGD) algorithm has been shown to consistently find optimal or near-optimal solutions to a variety of non-convex problems, particularly in machine learning [42]. We use a variant of SGD based on the score function estimator [43] to solve Equation 6. We randomly initialize a parameter matrix, ϕ⁽⁰⁾, with independent Normal samples, and then update the parameters according to

$${ϕ}^{( t )} = {ϕ}^{( t - 1 )} + α {∇}_{ϕ} F ( {ϕ}^{( t - 1 )} ) \qquad ( Eqn . 7 )$$

for t=1, . . . , T, where we define F(ϕ):= [symbol] _{q}_{_{ϕ}}₍ₓ₎[f(x)]+λH[q_{ϕ}] to be the objective function in Equation 6. The number of iterations, T, was set such that we observed convergence of the objective function values in most runs of the optimization.

After T iterations, we assumed that we had reached a near-optimal solution (i.e., ϕ^{(T)} can be used as an approximation of ϕ_{λ}). The components of the gradient in Equation 7 are given by

$$\begin{matrix}\frac{\partial }{\partial {ϕ}_{jk}} F ( ϕ ) = {𝔼}_{{q}_{ϕ} ( x )} [ w ( x ) \frac{\partial }{\partial {ϕ}_{jk}} log {q}_{{ϕ}_{j}} ( {x}^{j} ) ] \\ = {𝔼}_{{q}_{ϕ} ( x )} [ w ( x ) ( {δ}_{k} ( {x}^{j} ) - {q}_{{ϕ}_{j}} ( k ) ) ] ,\end{matrix} \qquad ( Eqn . 8 )$$

where we define the weights w(x):=f(x)−λ(1+log q_{ϕ}(x)). The expectation in Equation 8 cannot be solved exactly but can be solved numerically. In one implementation, we use a Monte Carlo approximation:

$$\frac{\partial }{\partial {ϕ}_{jk}} F ( ϕ ) \approx \frac{1}{M} {\sum }_{i = 1}^{M} w ( {x}_{i} ) ( {δ}_{k} ( {x}_{i}^{j} ) - {q}_{{ϕ}_{j}} ( {x}_{i}^{j} ) ) , \qquad {x}_{i} \sim {q}_{ϕ} ( x ) ,$$

where M is the number of samples used for the MC approximation. We applied this maximum entropy framework to design site-specific marginal probability libraries of the 21 nucleotides corresponding to the 7 amino acid insertion using the (NN, 100) predictive model of fitness. FIG. 8A shows the near-optimal Pareto frontier resulting from 2,238 such library optimizations with α=0.01, T=2000, and M=1000 and a range of settings of λ.

To solve the non-convex objective (Equation 6) for the library parameters, ϕ, some embodiments can use the Stochastic Gradient Descent (SGD) algorithm, which requires computing the gradient

$${∇}_{ϕ} [ {𝔼}_{{q}_{ϕ} ( x )} [ f ( x ) ] + λ H [ {q}_{ϕ} ] ] .$$

The gradient of the entropy is given by

$$\begin{matrix}{∇}_{ϕ} H [ {q}_{ϕ} ] = - {∇}_{ϕ} {𝔼}_{{q}_{ϕ} ( x )} [ log {q}_{ϕ} ( x ) ] \\ = - {\sum }_{x ∈ χ} {∇}_{ϕ} [ {q}_{ϕ} ( x ) log {q}_{ϕ} ( x ) ] \\ = - {\sum }_{x ∈ χ} ( log {q}_{ϕ} ( x ) {∇}_{ϕ} {q}_{ϕ} ( x ) + {q}_{ϕ} ( x ) {∇}_{ϕ} log {q}_{ϕ} ( x ) ) \\ = - {\sum }_{x ∈ χ} ( log {q}_{ϕ} ( x ) {q}_{ϕ} ( x ) {∇}_{ϕ} log {q}_{ϕ} ( x ) + {q}_{ϕ} ( x ) {∇}_{ϕ} log {q}_{ϕ} ( x ) ) \\ = - {\sum }_{x ∈ χ} {q}_{ϕ} ( x ) {∇}_{ϕ} log {q}_{ϕ} ( x ) ( 1 + log {q}_{ϕ} ( x ) ) \\ = - {𝔼}_{{q}_{ϕ} ( x )} [ ( 1 + log {q}_{ϕ} ( x ) ) {∇}_{ϕ} log {q}_{ϕ} ( x ) ]\end{matrix}$$

where in the third line we used the equality ∇_{ϕ}q_{ϕ}(x)=q_{ϕ}(x)∇_{ϕ} log q_{ϕ}(x). For ∇_{ϕ} [symbol] _{q}_{_{ϕ}}₍ₓ₎[f(x)], we use the equality ∇_{ϕ} [symbol] _{q}_{_{ϕ}}₍ₓ₎[f(x)] [symbol] _{q}_{_{ϕ}}₍ₓ₎)[f(x)∇_{ϕ} log q_{ϕ}(x)] which is well-known from its use in the score function estimator [1] (sometimes also called the ‘log derivative trick’). We then have

$${∇}_{ϕ} [ {𝔼}_{{q}_{ϕ} ( x )} [ f ( x ) ] + λ H [ {q}_{ϕ} ] ] = {∇}_{ϕ} {𝔼}_{{q}_{ϕ} ( x )} [ f ( x ) ] + λ {∇}_{ϕ} H [ {q}_{ϕ} ] = {𝔼}_{{q}_{ϕ} ( x )} [ f ( x ) {∇}_{ϕ} log {q}_{ϕ} ( x ) ] - {λ𝔼}_{{q}_{ϕ} ( x )} [ ( 1 + log {q}_{ϕ} ( x ) ) {∇}_{ϕ} log {q}_{ϕ} ( x ) ] = {𝔼}_{{q}_{ϕ} ( x )} [ w ( x ) {∇}_{ϕ} log {q}_{ϕ} ( x ) ] \qquad ( Eqn . S1 )$$

where w(x):=f(x)−λ(1+log q_{ϕ}(x)). The individual components of ∇_{ϕ} (log q_{ϕ}(x) are given by

$$\begin{matrix}\frac{\partial }{\partial {ϕ}_{jk}} log {q}_{ϕ} ( x ) = \frac{\partial }{\partial {ϕ}_{jk}} log {q}_{{ϕ}_{j}} ( {x}^{j} ) \\ = \frac{\partial }{\partial {ϕ}_{jk}} log \frac{{e}^{{ϕ}_{j , {x}^{j}}}}{{\sum }_{l = 1}^{K} {e}^{ϕ jl}} \\ = \frac{\partial }{\partial {ϕ}_{jk}} {ϕ}_{j , {x}^{j}} - \frac{\partial }{\partial {ϕ}_{jk}} log {\sum }_{l = 1}^{K} {e}^{ϕ jl} \\ = {δ}_{k} ( {x}^{j} ) - \frac{1}{{\sum }_{l = 1}^{K} {e}^{ϕ jl}} \frac{\partial }{\partial {ϕ}_{jk}} {\sum }_{l = 1}^{K} {e}^{ϕ jl} \\ = {δ}_{k} ( {x}^{j} ) - \frac{{e}^{ϕ jk}}{{\sum }_{l = 1}^{K} {e}^{ϕ jl}} \\ = {δ}_{k} ( {x}^{j} ) - {q}_{{ϕ}_{j}} ( k )\end{matrix} \qquad ( Eqn . S2 )$$

Using Equation S2 within Equation S1 gives Equation 8.

### B. Unconstrained Library

In some embodiments, a library is created by directly selecting sequences (sequence set) as opposed to generating the sequence set by sampling probability distributions.

As mentioned earlier, a distribution set of a designed library can specify the 84 marginal probabilities of individual nucleotides at each position in the 21-bp insertion (e.g., as shown in tables 1 and 2). As another example, probabilities of amino acids in a 7-mer insertion can be specified for a total of 140 probabilities. These libraries can be considered as constrained to a particular probability distribution. But an optimal library design (e.g., accounting for diversity) can be used for any library construction method, such as one that specifies and synthesizes individual 21-bp nucleotide sequences to create a library.

Contrasting the constrained libraries are unconstrained libraries, which are constructed as a list of oligonucleotide sequences that comprise the library. We use the term “unconstrained” to refer to libraries that are designed with this construction method since individual synthesis offers the most control over sequences in the library. In contrast, a position-wise nucleotide specification strategy, such as the use of a distribution set as described above, cannot guarantee the inclusion of any particular sequence. We thus refer to libraries constructed in this manner as “constrained” libraries. We have focused our experiments on these constrained libraries because they are currently more cost-effective, and thus most widely used. Indeed, Weinstein et al. [34] showed that for a fixed cost, the use of a constrained library construction can yield orders of magnitude more promising leads in protein engineering than an unconstrained (individual synthesis) approach. Note that technically, a fully unconstrained library is the probability distribution itself, p_{λ}(x), and that in drawing samples from such a distribution, the resulting library becomes an approximation to the unconstrained library in the sense of having only finitely many samples.

### 1. Diversity for Unconstrained Libraries

Entropy is closely related to another form of diversity known as effective sample size. The effective sample size of a library with entropy H is defined as Nₑ=e^{H}, and corresponds to how many unique variants one would need to obtain entropy H, if each variant was constrained to have equal probability mass. This can be seen by noting that

$$H = log {N}_{e} = - {\sum }_{i = 1}^{{N}_{e}} \frac{1}{{N}_{e}} log \frac{1}{{N}_{e}} .$$

This interpretation of entropy is commonly used in the population genetics literature, first introduced by S Wright in 1931 [44].

When comparing designed theoretical libraries, we were able to compute the statistical entropy of each library distribution exactly in terms of its position-wise probabilities. However, when analyzing post-selection libraries, there is no known underlying probability distribution with which we can exactly compute entropy. Consequently, we instead estimated and compared the effective sample size of the empirically observed distribution in each library. Specifically, we estimated the effective number of samples in a library using the sequencing observations:

$${N}_{e} = exp ( {\sum }_{s} - {p}_{empirical} ( s ) log - {p}_{empirical} ( s ) ) ,$$

where P_{empirical}(s) corresponds to the empirical frequency of sequence s appearing in the post-selection sequencing data.

### 2. Example Results for Unconstrained Libraries

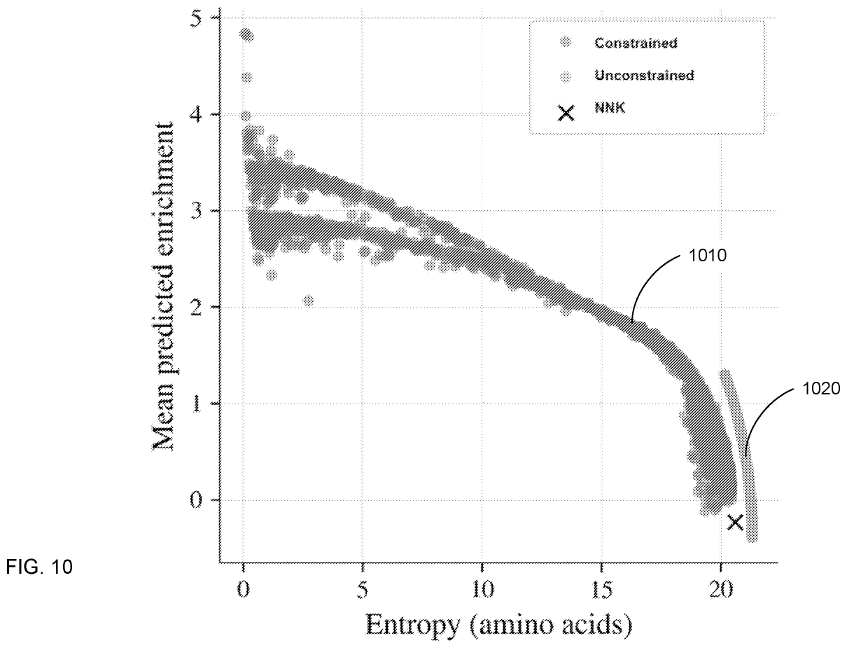

As the cost of individual synthesis declines, it will become increasingly useful to use our design approach to specify unconstrained libraries that are both diverse and have high fitness. With this future in mind, we also estimated the Pareto frontier for an unconstrained library (FIG. 10), which shows that, if cost were no concern, it would be advantageous to use an individual synthesis library construction approach, as its frontier substantially dominates that of a constrained library (i.e., use a distribution over sequences parameterized so that is completely unconstrained).

FIG. 10 shows the entropy and mean predicted log enrichment for constrained libraries and unconstrained libraries corresponding to 404 different settings of λ. Blue points 1010 are identical to the points in FIG. 8A, with the colors removed. Orange points 1020 represent unconstrained libraries defined by the distribution in Equation 4 with the same k values as used to construct the constrained libraries. The unconstrained libraries each include 10,000 sequences.

We can see that unconstrained library construction allows one to build a library with greater diversity at the same level of predicted fitness as constrained libraries. As oligonucleotide synthesis becomes cheaper, unconstrained library synthesis will became correspondingly cheaper. Therefore, our results suggest that at some point, it is likely that unconstrained libraries will become the libraries of choice.

Although we did not experimentally test any constrained libraries in this work, FIG. 10 demonstrates that it is possible to apply an optimized design (e.g., a maximum entropy formulation) to unconstrained libraries. For the purposes of comparison, we exactly compute the entropy and mean predicted log enrichment of the maximum entropy library defined by Equation 4 by enumerating all possible 7-mer insert sequences and evaluating the same predictive model, f(x), used to design the constrained libraries of FIG. 8A for each sequence. In general, even when it is not possible to fully enumerate the relevant sequence space, it is conceptually straightforward to build a list of sequences that approximates the maximum entropy library by sampling from this distribution with, for instance, Markov Chain Monte Carlo (MCMC) algorithms. This resulting set of samples represents a particle-based approximation to Equation 4 and thus approximately respect the Pareto optimal property of the maximum entropy library.

These results show that (i) we can build accurate predictive models for AAV packaging fitness for 7-mer insertion libraries; (ii) we can leverage these predictive models to design libraries that optimally trade off diversity with packaging fitness; and (iii) these designed libraries can be better starting libraries for downstream selection than standard libraries used today, despite not being tailored to the downstream task. The machine-learning based design to systematically identify a suite of optimal libraries along a trade-off curve of diversity and fitness.

### C. Flowchart for Optimizing a Library Based on Fitness and Diversity

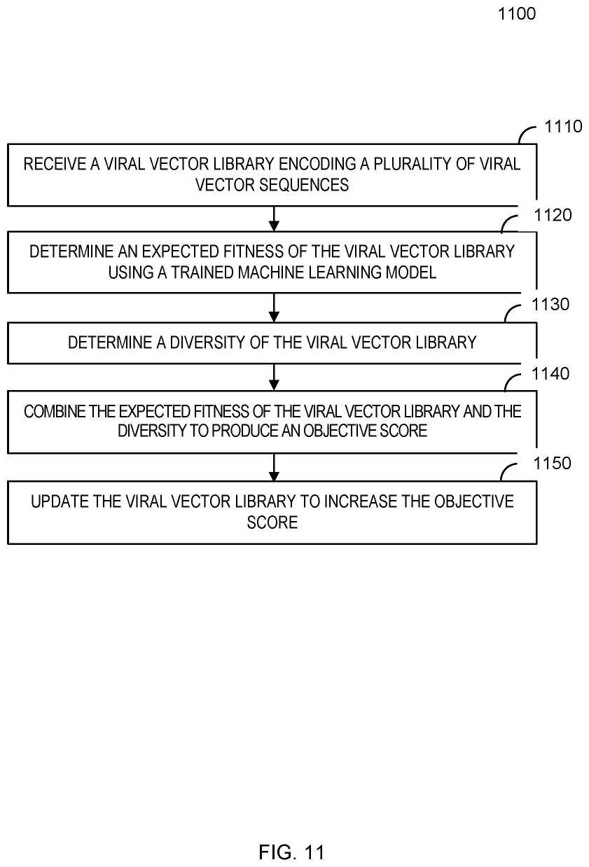

FIG. 11 shows a flowchart of an exemplary method of viral vector library design using a machine learning model. The trained machine learning model employed in method 1100 may comprise one of the herein disclosed machine learning architectures, trained according to one or more of the steps of the methods disclosed herein. Method 1100 may be executed by one or more of the systems discussed above. In some embodiments, method 1100 may be implemented by computing system 100 shown in FIG. 1. In some embodiments, method 1100 may be implemented based on instructions stored in library design module 110.

At operation 1110, the computing system receives a viral vector library encoding a plurality of viral vector sequences. In one example, a viral vector library may comprise a plurality of probability distributions, one for each of a pre-determined number of residues of a sequence. Each probability distribution can provide the probability of a current residue belonging to one of a fixed number of residue types/classes (e.g., 4 nucleotide classes or 20 amino acid classes). As an example, a three amino acid long viral vector library may comprise three probability distributions; each probability distribution provides, for a respective residue of the three-residue long sequence, a probability of the current residue being each of the 20 distinct amino acids. In this way, a large number of viral vector sequences may be efficiently encoded as a set or series of probability distributions. In other embodiments, the encoding may be achieved by a specific recitation of particular sequences, e.g., for an unconstrained library.

In some embodiments, a viral vector library may be initialized as the maximum entropy distribution for each residue, where each residue type is given an equal probability. In some embodiments, method 1100 may be performed iteratively to converge on a library design with a desired objective score, and in such embodiments the updated library produced by a previous iteration of method 1100 may be selected as the current viral vector library at operation 1102 of the current iteration.

At operation 1120, the computing system determines an expected library fitness (e.g., for packaging) of the viral vector library using a trained machine learning model, e.g., as described in section III. The library fitness is a collective fitness for the viral vector sequences in the library. For example, a library fitness value (e.g., an expected or experimentally measured library fitness value) can be a statistical value (e.g., an average, mean, mode, or median) representative of a characteristic of individual viral vector sequences in the viral vector library. Accordingly, determining the expected library fitness value can comprises determining expected fitness values for the individual viral vector sequences in the viral vector library and determining an average of the expected fitness values. Thus, a fitness can be determined for each of a plurality of viral vector sequences.

In some embodiments, determining the expected fitness of the viral vector library using the trained machine learning model comprises, mapping each of the plurality of viral vector sequences to a corresponding plurality of fitness values using the trained machine learning model. Each of the plurality of fitness values can be weighted based on a probability of occurrence of a corresponding viral vector sequence in the viral vector library to produce a plurality of weighted fitness values. The plurality of fitness values (e.g., weighted values) can be aggregated to produce the expected fitness of the viral vector library.

In some embodiments, if method 1100 is run iteratively to converge on a library design, the fitness values for each of the plurality of viral vector sequences of the current library may have been previously determined. In such embodiments, operation 1120 may comprise retrieving the previously determined fitness values of each of the plurality of viral vector sequences, weighting the plurality of fitness values based on the updated probabilities of each sequence occurring in the current library, and combining the weighted fitness values (e.g., by determining a statistical value) to produce the expected fitness value of the current library design.

If at operation 1120 the fitness values for the plurality of viral vector sequences has not been previously determined, the computing system may proceed to map each of the plurality of viral vector sequences to a corresponding fitness, using the trained machine learning model. The computing system may then weight the plurality of fitness values based on a corresponding probability of an associated viral vector sequence, to produce a plurality of weighted fitness values, and combine the plurality of weighted fitness values to determine the expected fitness value of the current viral vector library.

At operation 1130, the computing system determines a diversity of the viral vector library. In some embodiments, the diversity of the library may be based on the entropy of the plurality of probability distributions comprising the library. In one example, the diversity of the viral vector library may be determined according to the following equation:

$$H [ {q}_{ϕ} ] = - {\sum }_{i = 1}^{N} P ( {x}_{i} ) log P ( {x}_{i} )$$

where H[q_{ϕ}] is the diversity of the viral vector library, N is a total number of the plurality of viral vector sequences, i is an index over the plurality of viral vector sequences, and P(xᵢ) is a probability of occurrence of viral vector sequence i in the viral vector library.

In other embodiments (e.g., using an unconstrained library), diversity can be determined as an effective sample size, as described herein. Accordingly, some embodiments can be used for various library construction techniques, such as individual gene sequence specification and synthesis, as discussed above and as shown in FIG. 10.

At operation 1140, the computing system combines the expected fitness of the viral vector library and the diversity to produce an objective score. In some embodiments, the computing system may combine the diversity and expected fitness by weighting the diversity by a diversity trade-off factor to produce a weighted diversity, and adding the weighted diversity with the expected fitness to produce the objective score. In one example, the objective score may be given according to the following equation:

$$ℱ ( ϕ ) = {𝔼}_{{q}_{ϕ} ( x )} [ f ( x ) ] + λ H [ {q}_{ϕ} ]$$

wherein [symbol] (ϕ) is the objective score of the current library, [symbol] _{q}_{_{ϕ}}₍ₓ₎[f(x)] is the expected fitness [symbol] of the library, H [q_{ϕ}] is the diversity of the library, and A is the diversity trade-off factor.

At operation 1150, the computing system updates the viral vector library to increase the objective score. In some embodiments, the computing system may update the probability distributions encoding the plurality of viral vector sequences of the library based on a gradient descent algorithm. For example, the gradient of each probability distribution with respect to the objective score can be determined, and a step along the gradient of each probability can be taken. In one example, the library may be updated based on the following equation:

$${P}_{ij + 1} = {P}_{ij} + Step \frac{\partial ℱ}{\partial {P}_{ij}}$$

where Pᵢⱼ₊₁ is the updated probability of residue i being residue type j, Pᵢⱼ is the previous probability of residue i being residue type j, Step is the step size, and

$$\frac{\partial ℱ}{\partial {P}_{ij}}$$

is the partial derivative of the objective function [symbol] with respect to the previous probability.

If the viral vector library is determined in an unconstrained manner, the library may be updated by selecting a different set of viral vector sequences, e.g., ones that have a higher value for the combined score of expected fitness and diversity, potentially for any desired weighting of the diversity factor.

It will be appreciated that method 1100 may be run iteratively, until one or more convergence criteria are met. In one example, a convergence criteria may comprise a rate of change between an objective score of current library, and an objective score of a previous library, decreasing to below a threshold. In some embodiments, when a convergence criteria is not met, the updated library may be passed to operation 1110, and operations 1110-1150 may be executed using the updated library.

Various embodiments can also be extended to design libraries with other or multiple desired properties beyond packaging fitness and/or diversity. For instance, other properties/characteristics (e.g., cell sensitivity and specificity) can be used to select viral vector sequences for a library, alternatively or in addition to packaging fitness and/or diversity. For example, embodiments can replace the predictive model with one trained to simultaneously predict a different type of fitness value or multiple types of fitness values. As another example, separate predictive models can be used to independently predict fitness values for different properties/characteristics. This could be particularly useful to design libraries with improved cell sensitivity and specificity, which is particularly challenging using conventional experiment approaches.

Once a library of viral vector sequences is determined, embodiments can synthesize nucleic acids having viral vector sequences corresponding to the updated viral vector library. Once a nucleic acid having a viral vector sequence corresponding to the updated viral vector library is synthesized, the nucleic acid can be used to generate a packaged virus comprising a gene therapy. A subject can then be treated with the packaged virus.

### V. Performance of Library in Downstream Selection

Having demonstrated our ability to design and construct libraries with better packaging and good diversity, we next investigated how these gains would translate into performance on a downstream selection task for which the library had not been tailored. A goal was to design a generally useful library, agnostic to the downstream selection goal. Accordingly, after a library of sequences are synthesized, the resulting viral particles can be used for downstream gene therapy techniques. For this purpose, we studied a ML-designed AAV library for primary brain tissue infection.

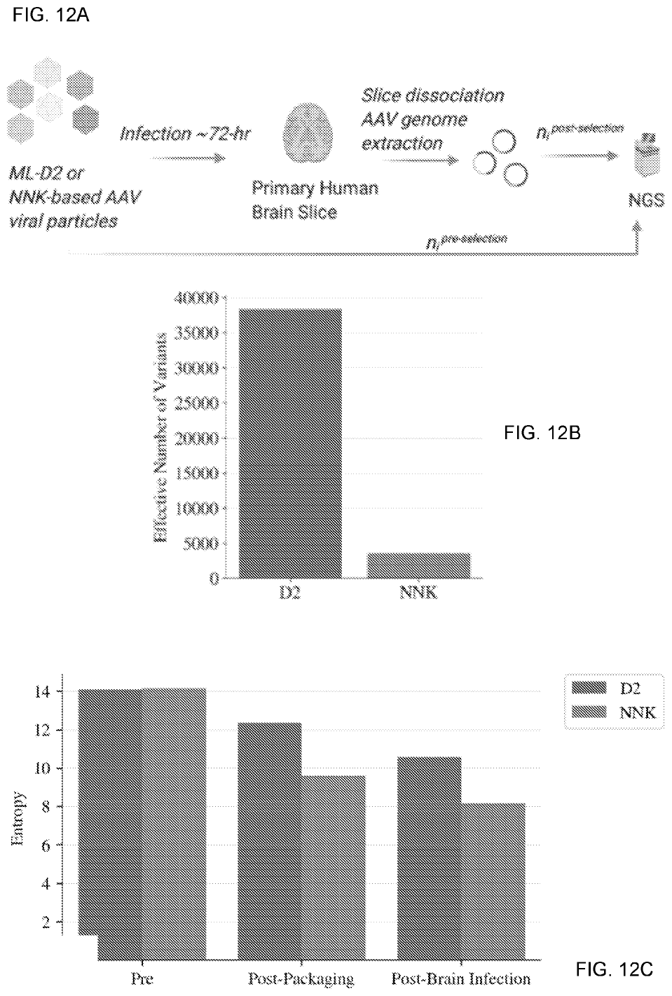

FIG. 12A shows a general workflow of the primary adult brain infection study. We used two of the libraries, the NNK as a baseline and our designed library D2 and used each to infect primary adult brain tissue. Infecting such tissue with AAV can be a first step toward numerous clinical applications in the central nervous system. We applied each library onto human adult brain slices and harvested the tissues after 72 hours of infection. We evaluated the success of each library on this task by comparing the effective number of variants in each pool after infectivity selection. A higher effective number of variants post-brain infection would suggest that the starting library contained more variants that were able to successfully infect human brain tissue, indicating a more useful starting library and larger set of promising variants.

FIG. 12B shows an effective number of variants (calculated from entropy) in NNK-post-brain infection vs. D2-post-brain infection. Both libraries started with similar entropy (diversity). We found that designed library D2 had a 10-fold higher post-brain infection effective number of variants than the NNK library—38,350 vs. 3,541 effective variants. This illustrates the better packaging of D2 over NNK.

FIG. 12C shows entropy (diversity) comparison between synthesized NNK and ML-designed D2 libraries after packaging and infection of primary adult brain tissue. D2 presents a comparable level of initial diversity (pre) to that of NNK but outperforms NNK after both packaging (post-packaging) and primary brain infection (post-brain-infection).

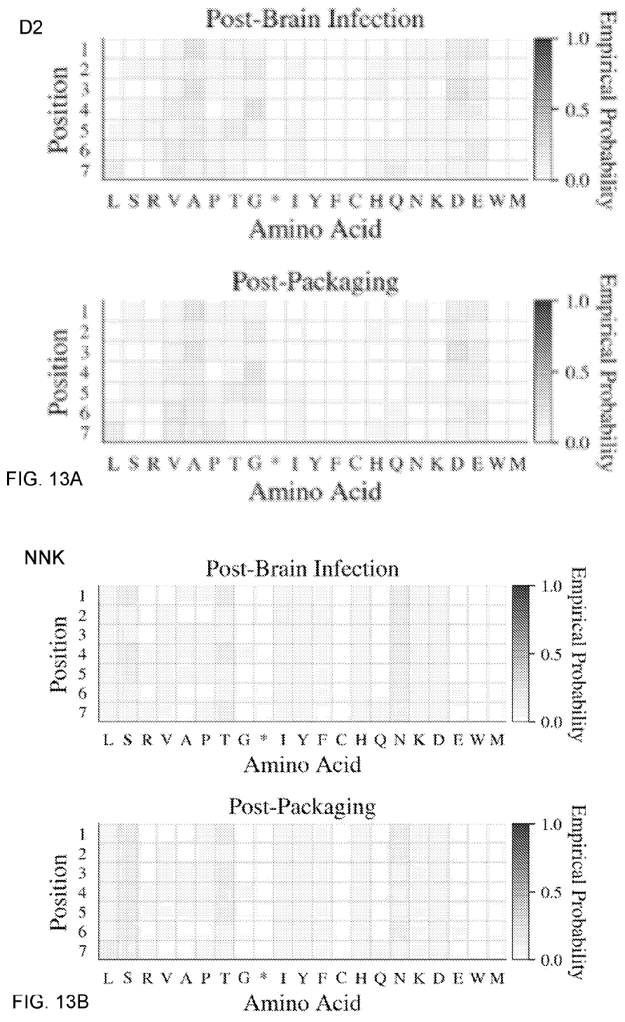

FIG. 13A shows empirical probabilities distribution of each amino acid at each position for D2 post-packaging and post-brain infection. Diversity can be achieved in different ways, and we were interested to know whether diversity was spread out over the length of the 7-mer insertion, or if some positions might have “collapsed” to be more constrained as a result of the selection. Therefore, for each post-packaging and post-brain infection library, we computed the probability of each amino acid at each position and found largely uniform distributions over amino acids, thereby revealing that position-wise diversity was well-maintained.

FIG. 13B shows NNK marginal probabilities of amino acids at each position after packaging and primary brain selection. The NNK marginal probabilities are also relatively uniform, but has much less packaging capability.

We next compared the post-packaging and post-brain infection libraries at the level of individual variants to assess some practical implications of the difference in diversity between the NNK and D2 libraries.

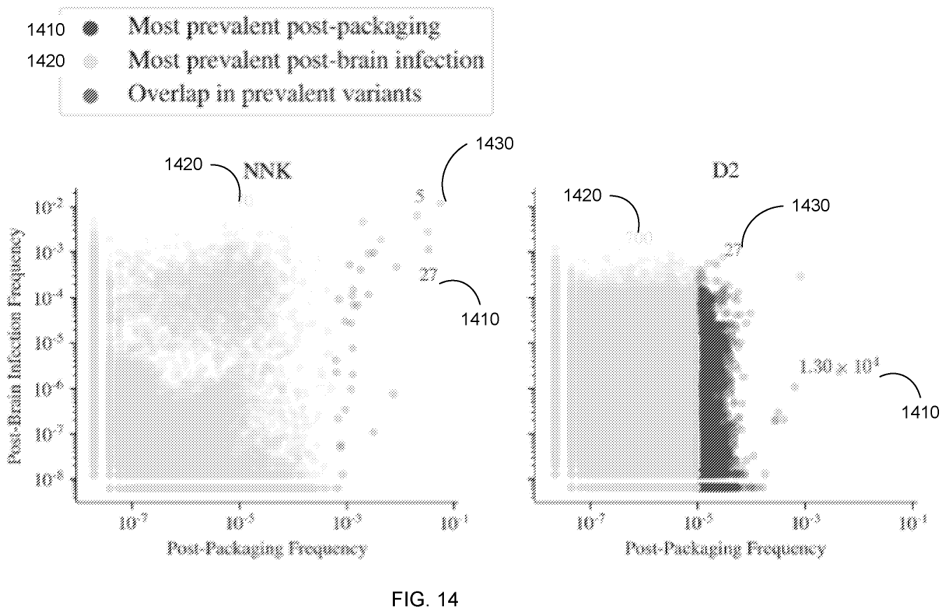

FIG. 14 shows scatterplots illustrating the behavior of individual variants over packaging and primary brain selection. Each axis shows the (log) prevalence of the variant in each library, as a fraction of reads in the library. For each library, variants in the top 20% are determined by first sorting unique variants by read count in descending order and then counting the number of unique variants comprising 20% of the total sequencing reads. Variants 1410 in the top 20% after packaging are colored blue, while variants 1420 in the top 20% after brain selection are colored yellow. Those variants 1430 in the top 20% of both packaging and selection are colored green. The annotated colored numbers indicate the number of variants of each colored pool. A pseudocount of 1 was added to each variant in each library prior to plotting.

We found a small set of variants dominated the post-packaging NNK library: the 32 most prevalent variants post-packaging (blue and green points in FIG. 14) accounted for 20% of the total sequencing reads. There were roughly 100-fold more unique variants in the top 20% of the D2 library post-packaging (1.32×10⁴ blue and green points), meaning that there is a much larger set of variants in the D2 library that package well when compared to the NNK library. In terms of downstream selection, the post-brain infection NNK library is dominated by a much smaller set of variants (˜10-fold fewer) compared to D2 (75 yellow and green points for NNK compared to 727 for D2). This suggests that the chances of discovering individual variants that successfully package and pass downstream selection are increased by using D2 instead of NNK as the starting library. In practice, then, the higher entropy for D2 post-packaging and post-brain infection translates to a much larger set of promising individual variants after each type of selection.

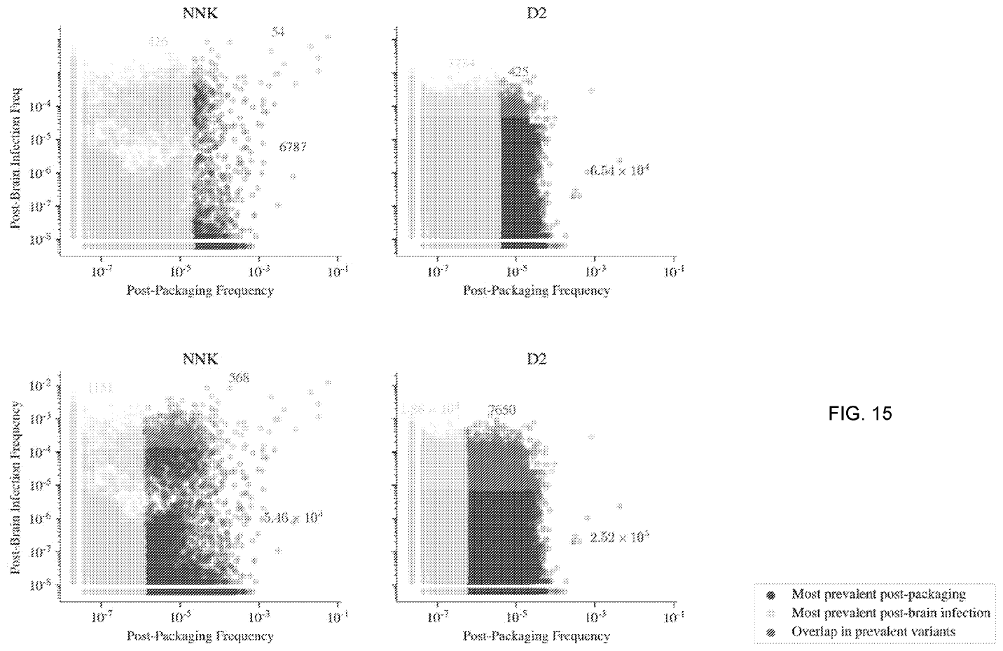

FIG. 15 shows scatterplots of individual variants' (log) prevalence after packaging and primary brain selections, displaying variants in the top 50% and 80% of each library. We also considered the top 50% and 80% of the post-packaging and post-brain infection libraries and found these conclusions to be consistent.

Collectively, the results demonstrate that our designed library D2 provided more useful diversity over the widely used NNK library, thereby making it an effective, general starting library for downstream selections for which it was not specifically designed.

We also validated that individual AAV variants from the ML-designed library D2 can not only package well but also successfully mediate cell-specific infection, which is a significant challenge in AAV engineering. For example, glial cells are important regulators of many aspects of human brain functions and diseases; however, true glial-cell specific targeting AAVs remain elusive [35]. To identify top variants for cell-specific expression validation, we applied the D2 library to human brain tissue, dissociated and isolated glial cells, extracted the cells' AAV genomes, and applied NGS.

We ranked the variants in the D2-post-glia infection library by the enrichment score (computed between the initial D2 library and D2-post-glial infection library) and selected the top variants for individual validation. Each of these top selected glial specific AAV variants showed high titers (˜10×10¹² vg/μL) when packaged with a GFP-encoding genome (Table 3).

TABLE 3
Experimental viral titers of glia-
infectious AAV variants from ML-D2.

| Sequences | Experimental Viral Titer (vg/μL) |
|---|---|
| Variant 1 | 2.84 × 10¹² |
| Variant 2 | 5.97 × 10¹² |
| Variant 3 | 9.14 × 10¹² |

Furthermore, immunostaining showed high levels of glial infection across multiple regions of the primary brain tissue.

The library design and selections can be extended to other cell types in brain or other tissues for a variety of therapeutic applications. For example, embodiments can be used for various downstream selection tasks, including those relevant to gene replacement in the nervous system and evasion of pre-existing antibodies.

Additional details about the brain analysis are provided below.

### A. Primary Human Adult Brain Slices Culture and Library Infection

Adult surgical specimens from epilepsy cases were obtained from the UCSF medical center in collaboration with neurosurgeons with previous patient consent. Surgically excised specimens were immediately placed in a sterile container filled with N-methyl-D-glucamine (NMDG) substituted artificial cerebrospinal fluid (aCSF) of the following composition (in mM): 92 NMDG, 2.5 KCl, 1.25 NaH₂PO₄, 30 NaHCO₃, 20 4-(2-hydroxyethyl)-1-piperazineethanesulfonic acid (HEPES), 25 glucose, 2 thiourea, 5 Na-ascorbate, 3 Na-pyruvate, 0.5 CaCl₂·4H₂O and 10 MgSO₄·7H₂O. The pH of the NMDG aCSF was titrated pH to 7.3-7.4 with 1M Tris-Base at pH8, and the osmolality was 300-305 mOsmoles/Kg. The solution was pre-chilled to 2-4° C. and thoroughly bubbled with carbogen (95% O₂/5% CO₂) gas prior to collection. The tissue was transported from the operating room to the laboratory for processing within 40-60 min. Blood vessels and meninges were removed from the cortical tissue, and then the tissue block was secured for cutting using superglue and sectioned perpendicular to the cortical plate to 300 μm using a Leica VT1200S vibrating blade microtome in aCSF. The slices were then transferred into a container of sterile-filtered NMDG aCSF that was pre-warmed to 32-34° C. and continuously bubbled with carbogen gas. After 12 min recovery incubation, slices were transferred to slice culture inserts (Millicell, PICM03050) on six-well culture plates (Corning) and cultured in adult brain slice culture medium containing 840 mg MEM Eagle medium with Hanks salts and 2 mM L-glutamine (Sigma, M4642), 18 mg ascorbic acid (Sigma, A7506), 3 mL HEPES (1M stock) (Sigma, H3537), 1.68 mL NaHCO₃ (892.75 mM solution, Gibco, 25080-094), 1.126 mL D-glucose, (1.11M solution, Gibco, A24940-01), 0.5 mL penicillin/streptomycin, 0.25 mL GlutaMax (at 400×, Gibco, 35050-061), 100 μL 2M stock MgSO₄·7H₂O (Sigma, M1880), 50 μL 2M stock CaCl₂·2H₂O (Sigma, C7902), 50 μL insulin from bovine pancreas, (10 mg/mL, Sigma, I0516), 20 mL horse serum-heat inactivated, 95 mL MilliQ H₂O (as previously described [45]). The following day after plating, adult human brain slices were infected with the viral library at an estimated of 10,000 MOI (N=3 per group) based on the number of cells estimated per slice. Slices were cultured at the liquid-air interface created by the cell-culture insert in a 37° C. incubator at 5% CO₂ for 72 hours post infection.

### B. Slice Culture Dissociation, Cell Purification and Hirt Extraction

Seventy-two hours after infection with the viral library, cultured brain tissue slices were first rinsed with DPBS (Gibco, 14190250) twice and detached from the filters. Then mechanically minced to 1 mm² pieces and enzymatically digested with papain digestion kit (Worthington, LK003163) with the addition of DNase for 1 hr at 37° C. After the enzymatic digestion, tissue was mechanically triturated using fire-polished glass pipettes (Fisher Scientific, cat #13-678-6A), filtered through a 40 μm cell strainer (Corning 352340), pelleted at 300×g for 5 minutes and washed twice with DBPS. Following mechanical digestion, the slices were first treated with lysis buffer (10% SDS, 1M Tris-HCL, pH 7.4-8.0, and 0.5M EDTA, pH 8.0) with the addition of RNase A (Thermo Scientific, EN0531) for 60 min at 37° C. and proteinase K (New England Biolabs, P8107S) for 3 hours at 55° C. The enzymatically digested tissue homogenate was then proceeded to the Hirt column protocol as previously published [46].

### C. Primary Prenatal Brain Slices

Deidentified primary tissue samples were collected with previous patient consent in strict observance of the legal and institutional ethical regulations. Cortical brain tissue was immediately placed in a sterile conical tube filled with oxygenated artificial cerebrospinal fluid (aCSF) containing 125 mM NaCl, 2.5 mM KCl, 1 mM MgCl₂, 1 mM CaCl₂), and 1.25 mM NaH₂PO₄ bubbled with carbogen (95% O₂/5% CO₂). Blood vessels and meninges were removed from the cortical tissue, and then the tissue block was embedded in 3.5% low-melting-point agarose (Thermo Fisher, BP165-25) and sectioned perpendicular to the ventricle to 300 μm using a Leica VT1200S vibrating blade microtome in a sucrose protective aCSF containing 185 mM sucrose, 2.5 mM KCl, 1 mM MgCl₂, 2 mM CaCl₂), 1.25 mM NaH₂PO₄, 25 mM NaHCO₃, 25 mM d-(+)-glucose. Slices were transferred to slice culture inserts (Millicell, PICM03050) on six-well culture plates (Corning) and cultured in prenatal brain slice culture medium containing 66% (vol/vol) Eagle's basal medium, 25% (vol/vol) HBSS, 2% (vol/vol) B27, 1% N2 supplement, 1% penicillin/streptomycin and GlutaMax (Thermo Fisher). Slices were cultured in a 37° C. incubator at 5% C02, 8% O₂ at the liquid-air interface created by the cell-culture insert.

### D. Slice Dissociation and Cell Purification

Cultured brain slices were washed twice with DPBS (Gibco, 14190250), detached from the filters and enzymatically digested with papain digestion kit (Worthington, LK003163) with the addition of DNase for 30 mins at 37° C. Following enzymatic digestion, slices were mechanically triturated using a fire-polished glass pipette, filtered through a 40 μm cell strainer test tube (Corning 352235), pelleted at 300×g for 5 minutes and washed twice with DBPS.

Dissociated cells were resuspended in MACS buffer (DPBS with 1 mM EGTA and 0.5% BSA) with addition of DNAse and incubated with CD11b antibody (microglia) for 15 minutes on ice. After the incubation, cells were washed in a 10 ml of MACS buffer and loaded on LS columns (Miltenyi Biotec, 130-042-401) on the magnetic stand. Cells were washed 3 times with 3 ml of MACS buffer, then the column was removed from the magnetic field and microglia cells were eluted using 5 ml of MACS buffer. The flow-through cells were then gently prepared to separate out neurons using polysialylated-neural cell adhesion molecule (PSA-NCAM), and the flow-through cell population was used as glial-cell type. Cells were pelleted, re-suspended in 1 ml of culture media and counted.

### E. Immunofluorescence and Antibodies

Primary human brain slices were fixed on the filters in 4% PFA for 1 hour at room temperature and washed 3× with PBS for 5 mins each wash. Slices were carefully detached from the culture filter inserts and places into 12 well plates. Blocking and permeabilization were performed in a blocking solution consisting of 10% normal donkey serum, 1% Triton X-100, and 0.2% gelatin for 1 hour. Primary and secondary antibodies were diluted and incubated in the blocking solution. Prenatal brain slices were incubated with primary antibodies at 4° C. overnight, washed 3× with washing buffer (1% Triton X-100 in PBS). Adult brain slices were incubated with primary antibodies for two days and washed 3× with washing buffer (1% Triton X-100 in PBS). Slices were incubated with secondary antibodies in the blocking buffer at 4° C. overnight and washed with washing buffer for 5× for 10 mins each. Images were collected using Leica SP8 confocal system with 10× and 20× air objective and processed using ImageJ/Fiji and Affinity Designer software. Primary antibodies used in this study included: chicken GFAP (1:1,000, Abcam, ab4674), rabbit dsRed (1:250, Takara, 632496), and DAPI. Secondary antibodies were species-specific AlexaFluor secondary antibodies (1:2,000, ThermoFisher).

### VI. Use for Other Types of Biological Sequences

Techniques described herein can also be used to determine properties of other types of biological sequences (e.g., nucleic acid sequences) besides viral vector sequences, or even viral sequences, where a protein coding sequence has been inserted. Such other biological sequences can be a viral sequence, a bacterial sequence, or be a host sequence, such as of an animal (including human) or plant. And as described above, the insertion does not have to be contiguous, and thus could essentially be multiple inserted sequences (each being one or more nucleotides) that are analyzed collectively. Any of the properties for viral sequences could be determined for the other types of sequences. An example method is described below. Further, any of the example implementation described for the viral vector sequences can be used for the biological sequences of the other use cases.

In step 1, a training data pair can be selected. The training data pair can comprise a nucleic acid sequence and a measured property value of the nucleic acid sequence. The nucleic acid sequence can include a synthetic sequence that codes for a protein, e.g., as described herein. The synthetic sequence can be inserted into an existing genetic sequence (e.g., a genome) or can replace nucleotides in the genetic sequence. The measured property value can be associated with the synthetic sequence. For example, the measured property value can be determined from an experiment applied to a biomolecule including the protein (e.g., a viral particle as described above). Examples of such experiments are provided above.

In step 2, the nucleic acid sequence can be encoded as a feature set. Examples of the encoding are provided in previous sections.

In step 3, the feature set is mapping to a predicted property value of the sequence using a machine learning model. Examples of such mapping using the machine learning model are provided in previous sections.

In step 4, a loss value is determined based on a difference between the measured property value and the predicted value of the nucleic acid sequence. Examples of such loss values are provided in previous sections.

In step 5, parameters of the machine learning model are updated based on the loss value. Example parameters and updating techniques (e.g., optimization techniques) are provided in previous sections.

Similarly, the optimization of a library can be performed for biological sequences, such as nucleic acid sequences. An example method is described below. Further, any of the example implementation described for the viral vector sequences can be used for the biological sequences of the other use cases.

In step 1, a nucleic acid sequence library encoding a plurality of nucleic acid sequences is received. In some implementations, at least a portion of the plurality of the nucleic acid sequences include a synthetic sequence that codes for a protein. Examples of libraries and encoding are provided in previous sections.

In step 2, an expected library fitness value of the nucleic acid sequence library is determined using a trained machine learning model. In some implementations, the expected library fitness value can be determined as a measured property value of the plurality of nucleic acid sequences. The measured property value can be determined from an experiment applied to a biomolecule including the protein.

In step 3, a diversity of the nucleic acid sequence library is determined. Examples of determining diversity are provided in previous sections.

In step 4, the expected library fitness value of the nucleic acid sequence library and the diversity are combined to produce an objective score. Examples of objective scores are provided in previous sections.

In step 5, the nucleic acid sequence library is updated to increase the objective score. Examples of updating the library are provided in previous sections.

### VII. Example Systems

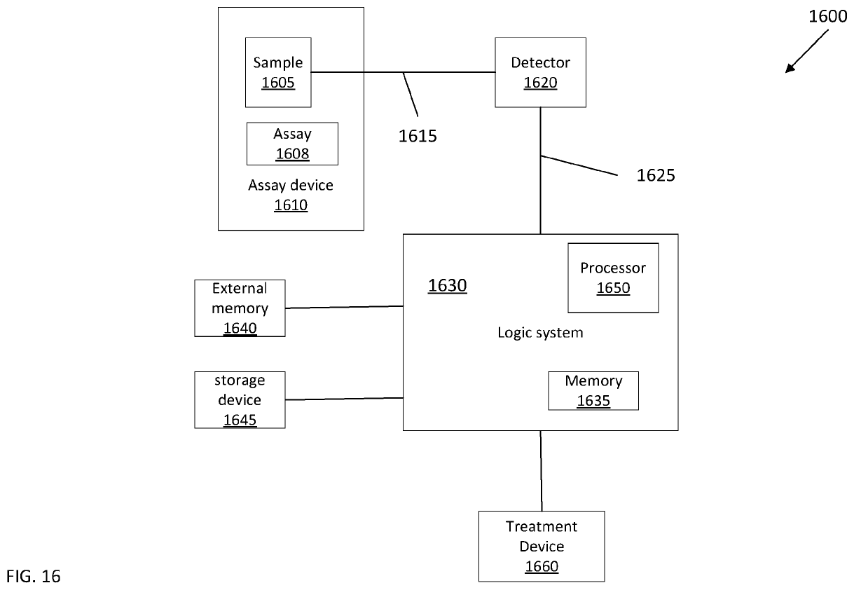

FIG. 16 illustrates a measurement system 1600 according to an embodiment of the present disclosure. The system as shown includes a sample 1605, such as cell-free DNA molecules within an assay device 1610, where an assay 1608 can be performed on sample 1605. For example, sample 1605 can be contacted with reagents of assay 1608 to provide a signal of a physical characteristic 1615. An example of an assay device can be a flow cell that includes probes and/or primers of an assay or a tube through which a droplet moves (with the droplet including the assay). Physical characteristic 1615 (e.g., a fluorescence intensity, a voltage, or a current), from the sample is detected by detector 1620. Detector 1620 can take a measurement at intervals (e.g., periodic intervals) to obtain data points that make up a data signal. In one embodiment, an analog-to-digital converter converts an analog signal from the detector into digital form at a plurality of times. Assay device 1610 and detector 1620 can form an assay system, e.g., a sequencing system that performs sequencing according to embodiments described herein. A data signal 1625 is sent from detector 1620 to logic system 1630. As an example, data signal 1625 can be used to determine sequences and/or locations in a reference genome of DNA molecules. Data signal 1625 can include various measurements made at a same time, e.g., different colors of fluorescent dyes or different electrical signals for different molecule of sample 1605, and thus data signal 1625 can correspond to multiple signals. Data signal 1625 may be stored in a local memory 1635, an external memory 1640, or a storage device 1645.

Logic system 1630 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 1630 and the other components may be part of a stand-alone or network connected computer system, or they may be directly attached to or incorporated in a device (e.g., a sequencing device) that includes detector 1620 and/or assay device 1610. Logic system 1630 may also include software that executes in a processor 1650. Logic system 1630 may include a computer readable medium storing instructions for controlling measurement system 1600 to perform any of the methods described herein. For example, logic system 1630 can provide commands to a system that includes assay device 1610 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 1600 may also include a treatment device 1660, which can provide a treatment to the subject. Treatment device 1660 can determine a treatment and/or be used to perform a treatment. Examples of such treatment can include surgery, radiation therapy, chemotherapy, immunotherapy, targeted therapy, hormone therapy, and stem cell transplant. Logic system 1630 may be connected to treatment device 1660, e.g., to provide results of a method described herein. The treatment device may receive inputs from other devices, such as an imaging device and user inputs (e.g., to control the treatment, such as controls over a robotic system).

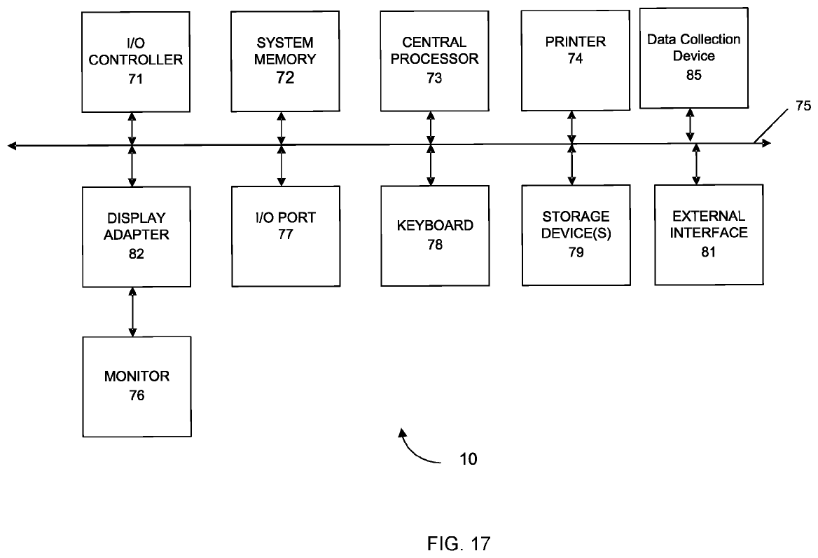

Any of the computer systems mentioned herein may utilize any suitable number of subsystems. Examples of such subsystems are shown in FIG. 17 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 17 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

1. A method comprising:
selecting a training data pair comprising a viral vector sequence and a ground truth fitness value of a characteristic of the viral vector sequence;
encoding the viral vector sequence as a feature set;
mapping the feature set to a predicted fitness value of the viral vector sequence using a machine learning model;
determining a loss value based on a difference between the ground truth fitness value and the predicted fitness value of the viral vector sequence; and
updating parameters of the machine learning model based on the loss value.

2. The method of claim 1, wherein the ground truth fitness value is an experimentally measured property of the viral vector sequence to deliver a gene therapy to a cell.

3. The method of claim 1, wherein the characteristic is packaging of the viral vector sequence, wherein the ground truth fitness value is a ground truth packaging fitness value, and wherein the predicted fitness value is a predicted packaging fitness value.

4. The method of claim 3, wherein the ground truth packaging fitness value is a function of a first abundance of the viral vector sequence, measured before a packaging process, and a second abundance of the viral vector sequence, measured after the packaging process.

5. The method of claim 4, wherein the function is given as:
$${y}_{i} = log \frac{{n}_{i}^{post}}{{n}_{i}^{pre}} - log \frac{{N}^{post}}{{N}^{pre}} ,$$
wherein:
y_i is the ground truth packaging fitness of the viral vector sequence i;
nᵢ^{pre} is the first abundance of the viral vector sequence I measured before the packaging process;
nᵢ^{post} is the second abundance of the viral vector sequence i measured after the packaging process;
N^{pre} is a first total abundance of viral vector sequences measured before the packaging process; and
N^{post} is a second total abundance of viral vector sequences measured after the packaging process.

6. The method of claim 1, wherein encoding the viral vector sequence as the feature set comprises:
one-hot encoding each residue of the viral vector sequence to produce a plurality of independent site vectors.

7. The method of claim 1, wherein encoding the viral vector sequence as the feature set comprises:
encoding each pair of adjacent residues of the viral vector sequence as a vector to produce a plurality of interaction vectors.

8. The method of claim 1, wherein encoding the viral vector sequence as the feature set comprises:
encoding each residue-residue interaction of the viral vector sequence to produce a plurality of interaction vectors.

9-10. (canceled)

11. The method of claim 1, wherein determining the loss value based on the difference between the ground truth fitness value and the predicted fitness value of the viral vector sequence comprises:
determining an unweighted loss based on the difference between the ground truth fitness value and the predicted fitness value of the viral vector sequence; and
weighting the unweighted loss based on a variance of the viral vector sequence to produce the loss value.

12. The method of claim 11, wherein the characteristic is packaging of the viral vector sequence, wherein the variance is given by:
$${σ}_{i}^{2} = \frac{1}{{n}_{i}^{post}} ( 1 - \frac{{n}_{i}^{post}}{{N}^{post}} ) + \frac{1}{{n}_{i}^{pre}} ( 1 - \frac{{n}_{i}^{pre}}{{N}^{pre}} ) ,$$
wherein:
σᵢ² is the variance of the viral vector sequence i;
nᵢ^{pre} is a first abundance of the viral vector sequence measured before a packaging process;
nᵢ^{post} is a second abundance of the viral vector sequence measured after the packaging process;
N^{pre} is a first total abundance of viral vector sequences measured before the packaging process; and
N^{post} is a second total abundance of viral vector sequences measured after the packaging process.

13. A method comprising:
receiving a viral vector library encoding a plurality of viral vector sequences;
determining an expected library fitness value of the viral vector library using a trained machine learning model;
determining a diversity of the viral vector library;
combining the expected library fitness value of the viral vector library and the diversity to produce an objective score; and
updating the viral vector library to increase the objective score.

14. The method of claim 13, wherein the expected library fitness value is a statistical value representative of a characteristic of individual viral vector sequences in the viral vector library.

15. (canceled)

16. The method of claim 14, wherein the characteristic is packaging of the individual viral vector sequences.

17. The method of claim 13, further comprising:
synthesizing nucleic acid sequences corresponding to the updated viral vector library.

18. The method of claim 13, further comprising:
synthesizing a nucleic acid sequence corresponding to the updated viral vector library; and
generating, from the nucleic acid sequence, a packaged virus comprising a gene therapy.

19. The method of claim 18, further comprising:
treating a subject with the packaged virus.

20. The method of claim 13, wherein the viral vector library encodes the plurality of viral vector sequences as a pre-determined number of probability distributions over a pre-determined number of residues, wherein a probability distribution for a residue provides a probability of the residue being one of a fixed number of residue types.

21. The method of claim 20, wherein the diversity of the viral vector library is given by:
$$H [ {q}_{ϕ} ] = {\sum }_{i = 1}^{N} P ( {x}_{i} ) log P ( {x}_{i} ) ,$$
wherein:
H[q_{ϕ}] is the diversity of the viral vector library;
N is a total number of the plurality of viral vector sequences;
i is an index over the plurality of viral vector sequences; and
P(xᵢ) is a probability of occurrence of viral vector sequence i in the viral vector library.

22. The method of claim 20, wherein combining the expected library fitness value of the viral vector library and the diversity to produce the objective score comprises:
weighting the diversity by a diversity trade-off factor to produce a weighted diversity; and
adding the weighted diversity with the expected library fitness value to produce the objective score.

23. The method of claim 13, wherein the viral vector library encodes the plurality of viral vector sequences as a pre-determined probability distribution over a pre-determined number of residues, wherein a probability distribution for a sequence position provides a probability of a residue being one of a fixed number of types.

24-33. (canceled)
